(19) United States
(12) Patent Application Publication
(10) Pub. No.: US 2013/0095179 A1
(43) Pub. Date: Apr. 18, 2013

(54) METHODS AND COMPOSITIONS FOR TREATING, REVERSING, INHIBITING OR PREVENTING RESISTANCE TO ANTIPLATELET THERAPY

(71) Applicants: Michael H. Davidson, Highland Park, IL (US); Gerald L. Wisler, Windermere, FL (US)

(72) Inventors: Michael H. Davidson, Highland Park, IL (US); Gerald L. Wisler, Windermere, FL (US)

(73) Assignee: Omthera Pharmaceuticals, Inc., Princeton, NJ (US)

(21) Appl. No.: 13/620,312

(22) Filed: Sep. 14, 2012

Related U.S. Application Data
(60) Provisional application No. 61/535,192, filed on Sep. 15, 2011.
(60) Provisional application No. 61/549,907, filed on Oct. 21, 2011.

(51) Int. Cl. A61K 31/202; A61K 31/616; A61K 31/4365

(52) U.S. Cl. CPC A61K 31/202; A61K 31/4365; A61K 31/616
USPC 424/456; 514/560; 514/301; 514/165

(57) ABSTRACT

Methods of identifying subjects who are resistant to antiplatelet therapy, such as therapy with clopidogrel, are presented. The methods comprise determining is whether the subject is an efficient converter of medium chain polyunsaturated fatty acids to long-chain polyunsaturated fatty acids. Also provided are methods of treating resistance to antiplatelet therapy in subjects who are efficient converters of medium chain polyunsaturated fatty acids to long-chain polyunsaturated fatty acids, comprising adjunctively administering to the subject an effective amount of a composition comprising omega-3 long chain polyunsaturated fatty acids. Improved methods of antiplatelet therapy are provided, wherein the improvement comprises adjunctive administration of a composition comprising omega-3 long chain polyunsaturated fatty acids in free acid form. Dosage forms comprising at least one antiplatelet agent and compositions comprising omega-3 long chain polyunsaturated fatty acids, including compositions comprising omega-3 long chain polyunsaturated fatty acids in free acid form, are provided.

### 1. CROSS-REFERENCE TO RELATED APPLICATIONS

This application claims the benefit under 35 U.S.C. §119(e) of U.S. Provisional Application Nos. 61/535,192, filed Sep. 15, 2011 and 61/549,907, filed Oct. 21, 2011, the contents of both of which are incorporated herein in their entireties by reference thereto.

### 2. BACKGROUND

Clopidogrel bisulfate (Plavix®) is a platelet aggregation inhibitor administered to protect against fatal or non-fatal heart attack or stroke in patients with a history of heart attack, stroke, peripheral arterial disease or acute coronary syndrome. Despite its widespread use and clinical benefit, significant individual variability in platelet response to clopidogrel has been observed. Serebruany et al., 2005, J. Am. Coll. Cardiol. 45(2):246-251. It is estimated that between 4% and 30% of patients exhibit “clopidogrel resistance”, i.e., when treated with conventional doses of clopidogrel, they do not display adequate anti-platelet response. Nguyen et al., 2005, J. Am. Coll. Cardiol. 45(8):1157-64. Clopidogrel resistance has been found to increase the risk of recurrent cardiovascular events in certain subsets of patients. Nguyen et al., 2005, J. Am. Coll. Cardiol. 45(8):1157-64; Matetzky et al., 2004, Circulation 109:3171-75.

Polymorphisms in the gene encoding hepatic cytochrome P450 isozyme CYP2C19 have been found to be associated with diminished platelet response to clopidogrel in healthy subjects and in patients with coronary artery disease or who were undergoing cardiac intervention. Hulot et al., 2006, Blood 108(7):2244-47; Schulinder et al., 2009, JAMA 302(8):849-858. The loss of function polymorphisms in the 2C19 gene are associated with decreased conversion of clopidogrel to its active metabolite and poorer cardiovascular outcomes. Schulinder et al., 2009, JAMA 302(8):849-858; Pettersen et al., 2011, Thrombosis J. 9:4-11. In view of the compelling evidence that genetic variation in CYP 2C19 is a significant predictor of clinical response to clopidogrel therapy, the FDA has issued a warning that Plavix® could have reduced effectiveness in patients who are poor metabolizers.

U.S. Patent Publication Nos. 2011/0045481 and 2011/0060532 to Gladding et al. describe methods for predicting or determining a subject's response to antiplatelet therapy and methods of determining a subject's suitability to a treatment regime or intervention for a disease associated with platelet aggregation by analyzing CYP2C19 polymorphisms. U.S. Patent Publication No. 2011/0159479 to Industry-University Cooperation Foundation Yonsei University describes methods for predicting the resistance of a human subject to clopidogrel by detecting a polymorphism in CYP2C19.

Nevertheless, polymorphism in CYP genes is not the only factor that contributes to clopidogrel resistance, as one study found that 22% of patients without the CYP2C19*2 polymorphism were resistant, while about 50% of patients with the polymorphism were responders. Pettersen et al., 2011, Thrombosis J. 9:4-11.

There is, therefore, a need for compositions and methods for treating patients requiring inhibition of platelet aggregation (“antiplatelet therapy”) (such as therapy with clopidogrel and therapy with aspirin) that increase the efficacy of the antiplatelet therapies, especially in resistant subjects.

### 3. SUMMARY

The inventors have discovered that elevated plasma levels of arachidonic acid (“AA”) are associated with resistance to antiplatelet therapy in a subset of patients resistant to antiplatelet therapy; that elevated AA levels in certain such patients can be attributed to an enhanced ability to convert medium chain polyunsaturated fatty acids (“mc-PUFAs”) to long chain polyunsaturated fatty acids (“lc-PUFAs”); and that resistance to antiplatelet therapy in such efficient converters can be treated, reversed, inhibited, or prevented by treatment with compositions enriched in omega-3 lc-PUFAs. An efficient converter, as described in more detail below, is a subject who more efficiently produces long chain polyunsaturated fatty acid products from dietary medium chain fatty acids than a subject who is not an efficient converter.

The inventors have further discovered that compositions comprising omega-3 lc-PUFAs in free acid form (“n-3 FFA compositions”) provide unprecedented potency in reducing AA plasma levels. The exceptional potency allows such n-3 FFA compositions to be used to treat, reverse, inhibit or prevent resistance to antiplatelet therapy in efficient converters using clinically relevant doses. The high potency also allows such n-3 FFA compositions to be administered at that same or at reduced dosage as an adjunct to antiplatelet therapy in patients who are not efficient converters—both those with elevated plasma AA levels and those with average AA plasma levels—with the potent AA-lowering effect of the n-3 FFA compositions improving the efficacy of antiplatelet therapies in nearly all such patients.

Thus, in a first aspect, methods are presented for treating, reversing, inhibiting, or preventing resistance to antiplatelet therapy in a subject who is an efficient converter and for whom antiplatelet therapy is clinically indicated. The methods comprise administering to the subject an effective amount of a composition comprising omega-3 lc-PUFAs (“omega-3 composition”). In certain embodiments, the methods further comprise the prior step of determining whether the subject is an efficient converter.

In certain embodiments, determining whether the subject is an efficient converter comprises determining the subject's genotype at one or more polymorphisms associated with one or more genes selected from the group consisting of FADS1, FADS2, and FADS3. In various embodiments, determining whether the subject is an efficient converter comprises measuring the level of arachidonic acid in a sample from the subject.

In typical embodiments, the amount of omega-3 composition is effective to reduce arachidonic acid (AA) concentration in plasma by at least about 5%. In certain embodiments, the amount of omega-3 composition is effective to reduce plasma AA concentration by at least about 10%. In a series of embodiments, the amount of omega-3 composition is effective to reduce plasma AA concentration by at least about 20%.

In a variety of embodiments, the amount of omega-3 composition is effective to reduce plasma arachidonic acid concentration by at least about 50 μg/mL, even by at least about 75 μg/mL.

In a variety of embodiments, the amount of omega-3 composition is effective to increase plasma EPA/AA ratio to at least about 0.25, and in some embodiments, is effective to increase plasma EPA/AA ratio to at least about 0.50, even to at least about 0.65.

In certain embodiments, the polyunsaturated fatty acids are present in the composition in free acid form (“n-3 FFA compositions”). In various embodiments, the n-3 FFA composition comprises at least 50% EPA by area on GC chromatogram of all fatty acids in the composition (“50% (a/a)”). In various embodiments, the n-3 FFA composition further comprises at least 15% (a/a) DHA. In yet further embodiments, the n-3 FFA composition further comprises at least 2.5% (a/a) DPA.

In specified embodiments, the amount of omega-3 composition is no more than 4 g/day. In particular embodiments, the amount of omega-3 composition is no more than 2 g/day.

In another aspect, methods are presented for providing antiplatelet therapy to subjects in need thereof. The methods comprise (a) determining whether the subject is an efficient converter; and (b) in those subjects determined to be efficient converters, adjunctively administering (i) an effective amount of an omega-3 composition, and (ii) an effective amount of an antiplatelet agent.

In a related aspect, an improved method of providing antiplatelet therapy to subjects in need thereof is provided, wherein the improvement comprises (a) determining whether the subject is an efficient converter; and (b) in those subjects determined to be efficient converters of mc-PUFA to lc-PUFA, adjunctively administering an effective amount of an omega-3 composition.

In a variety of embodiments of these methods, determining whether the subject is an efficient converter comprises determining the subject's genotype at one or more polymorphisms associated with one or more genes selected from the group consisting of FADS1, FADS2, and FADS3. In certain embodiments, determining whether the subject is an efficient converter comprises measuring the level of arachidonic acid in a sample from the subject.

Embodiments of these methods include those in which the amount of omega-3 composition is effective to reduce arachidonic acid (AA) concentration in plasma by at least about 5%, by at least about 10%, and by at least about 20%. In certain embodiments, the amount of omega-3 composition is effective to reduce plasma arachidonic acid concentration by at least about 50 μg/mL, even by at least about 75 μg/mL. In various embodiments, the amount of omega-3 composition is effective to increase plasma EPA/AA ratio to at least about 0.25, to at least about 0.50, even to at least about 0.65.

In presently preferred embodiments, the omega-3 composition is an n-3 FFA composition. In certain embodiments, the n-3 FFA composition comprises at least 50% (a/a) EPA. In particular embodiments, the n-3 FFA composition further comprises at least 15% (a/a) DHA. In specific embodiments, the n-3 FFA composition further comprises at least 2.5% (a/a) DPA.

In embodiments of these methods, the amount of omega-3 composition is no more than 4 g/day. In particular embodiments, the amount of omega-3 composition is no more than 2 g/day.

The antiplatelet agent, in certain embodiments, is clopidogrel bisulfate or aspirin, or combinations thereof. In specific embodiments, the antiplatelet agent is clopidogrel bisulfate.

In another aspect, methods are presented for treating a patient with an antiplatelet agent. The methods comprise (a) administering a therapeutically effective amount of an inhibitor of platelet aggregation; and (b) adjunctively administering an effective amount of n-3 FFA composition. In a related aspect, improved methods of treating patients with antiplatelet therapy are provided, in which the improvement comprises adjunctively administering an effective amount of an n-3 FFA composition.

In certain embodiment, the amount of n-3 FFA composition is effective to reduce arachidonic acid (AA) concentration in plasma by at least about 5%, by at least about 10%, by at least 15%, by at least 20%, even by at least 25%.

The amount of n-3 FFA composition in various embodiments is effective to reduce plasma arachidonic acid concentration by at least about 10 μg/mL, at least about 15 μg/mL, at least about 20 μg/mL, and at least about 25 μg/mL. In particular embodiments, the amount of n-3 FFA composition is effective to reduce plasma arachidonic acid concentration by at least about 50 μg/mL, even at least about 75 μg/mL.

In certain embodiments, the amount of n-3 FFA composition is effective to increase plasma EPA/AA ratio to at least about 0.25, at least about 0.50, even at least about 0.65.

In presently preferred embodiments, the n-3 FFA composition comprises at least 50% (a/a) EPA. In certain embodiments, the n-3 FFA composition further comprises at least 15% (a/a) DHA. In particular embodiments, the n-3 FFA composition further comprises at least 2.5% (a/a) DPA. In a specific embodiment, the n-3 FFA composition comprises about 55% EPA (a/a), about 20% DHA (a/a), and about 5% DPA (a/a).

The methods, in some embodiments, comprise administering no more than 4 g/day of the n-3 FFA composition. In some embodiments, no more than 2 g/day is administered.

In typical embodiments, the antiplatelet agent is selected from the group consisting of clopidogrel bisulfate and aspirin, and in specific embodiments, the antiplatelet agent is clopidogrel bisulfate.

In a further aspect, a unit dosage form is presented. The unit dosage form comprises both an omega-3 composition and an anti-platelet agent. In typical embodiments, the omega-3 composition is contained within a capsule, and the anti-platelet agent is coated on the exterior of said capsule.

In typical embodiments, the anti-platelet agent is clopidogrel bisulfate or aspirin. In specific embodiments, the anti-platelet agent is clopidogrel bisulfate.

In various embodiments, at least 0.5 g of omega-3 composition is encapsulated in the unit dosage form. In certain embodiments, at least 1 g of omega-3 composition is encapsulated.

In presently preferred embodiments, the omega-3 composition encapsulated in the unit dosage form is an n-3 FFA composition. In typical embodiments, the n-3 FFA composition comprises at least 50% (a/a) EPA. In particular embodiments, the n-3 FFA composition further comprises at least 15% (a/a) DHA. In certain embodiments, the n-3 FFA composition further comprises at least 2.5% (a/a) DPA.

In a variety of embodiments, and particularly embodiments in which the omega-3 composition is an n-3 FFA composition, the capsule of the unit dosage form is a porcine type A soft gelatin capsule.

In certain embodiments, the capsule further comprises a coating interposed between the gelatin and the coating comprising the anti-platelet agent. The interposed coating, in typical such embodiments, is capable of delaying release of the n-3 FFA composition for at least 30 minutes at 37° C. in aqueous medium in vitro. In specific embodiments, the interposed coating is a neutral poly(ethylacrylate-methylmethacrylate) polymer.

The features and advantages of the disclosure will become further apparent from the accompanying drawings, and the following detailed description of embodiments thereof.

### 4. BRIEF DESCRIPTION OF THE FIGURES

FIG. 1 shows the known metabolic pathway of conversion of the dietary fatty acids linoleic acid (an omega-6 fatty acid) and α-linolenic acid (an omega-3 fatty acid) to long chain polyunsaturated fatty acids (“lc-PUFAs”) in the human body.

FIGS. 2-24 depict arachidonic acid (AA) plasma levels for subjects in the clinical trial further described in Example 2, grouped according to genotype at the respectively identified SNPs, at (A) baseline (in μg/mL), and (B) day 15 of treatment with an n-3 FFA composition (defined herein below) (in percent change from baseline). For each genotype, the interquartile range is indicated by a box, the median is indicated by a horizontal line in the interior of the interquartile box, and the mean is represented by a diamond. Outliers are represented by open circles. The whiskers extend to the minimum and maximum non-outlier value. Score 1 identifies subjects who are homozygous at the major allele; Score 3 identifies subjects homozygous at the minor allele; and Score 2 represents heterozygotes.

FIG. 25 is a bar chart of baseline and Day 15 AA levels (μg/mL) for each genotype at SNP rs174537 in the trial further described in Example 2.

FIG. 26 is a bar chart of baseline and end-of-treatment (“EOT”) EPA levels (μg/mL) for each genotype at SNP rs174546 in a clinical study of interaction of Epanova® and warfarin, where baseline EPA is the average of 7 pre-dose plasma concentration values, from Day 7 and Day 8 for the Warfarin/Epanova arm, and Day −1 and Day 1 for the Lovaza arm. End of Treatment EPA is the average of 3 pre-dose plasma concentration values, from Day 18, Day 19, and Day 20 for the Warfarin/Epanova arm, and Days 11, 12 and 13 for the Lovaza arm.

FIG. 27 provides a treatment flow diagram illustrating the design of the EVOLVE study, further described in Example 3.

FIG. 28 summarizes the EVOLVE trial design in greater detail, further identifying the timing of study visits.

FIG. 29 shows the disposition of subjects in the EVOLVE trial.

FIGS. 30A-30E display average baseline and end-of-treatment (“EOT”) plasma levels (in μg/mL) for EPA (FIG. 30A), DHA (FIG. 30B), DPA (FIG. 30C) and AA (FIG. 30D), for each of the treatment arms in the EVOLVE trial. FIG. 30E compares average baseline and EOT EPA levels for each treatment arm of the EVOLVE trial described in Example 3, the control (olive oil) arm of the EVOLVE trial described in Example 3, and values earlier reported in the literature for the unrelated JELIS trial (“JELIS”).

FIGS. 31A-31D plot median baseline and end-of-treatment (“EOT”) plasma levels (in μg/mL) for EPA (FIG. 31A), DHA (FIG. 31B), DPA (FIG. 31C), and AA (FIG. 31D).

FIGS. 32A and 32B plot change from baseline to EOT in absolute plasma levels (in μg/mL) of AA, DHA, EPA, and DPA, for each of the treatment arms of the EVOLVE trial, with FIG. 32A plotting average change and FIG. 32B showing median change from baseline.

FIG. 33A plots average change from baseline to EOT, as percentage of baseline value, for AA, DHA, EPA, and DPA in each of the treatment arms of the EVOLVE trial, with FIG. 33B plotting median percent change from baseline to EOT.

FIG. 34 plots the rate of change (absolute value of the slope) of the median percentage change from baseline in plasma levels of EPA, DHA, DPA, and AA between 2 g and 4 g doses of EPANOVA.

### 5. DETAILED DESCRIPTION

The inventors have discovered that elevated plasma levels of arachidonic acid (“AA”) are associated with resistance to antiplatelet therapy in a subset of patients resistant to antiplatelet therapy; that elevated AA levels in certain such patients can be attributed to an enhanced ability to convert medium chain polyunsaturated fatty acids (“mc-PUFAs”) to long chain polyunsaturated fatty acids (“lc-PUFAs”); and that resistance to antiplatelet therapy in such efficient converters can be treated, reversed, inhibited, or prevented by treatment with compositions enriched in omega-3 lc-PUFAs. An efficient converter, as described in more detail below, is a subject who more efficiently produces long chain polyunsaturated fatty acid products from dietary medium chain fatty acids than a subject who is not an efficient converter.

The inventors have further discovered that compositions comprising omega-3 lc-PUFAs in free acid form (“n-3 FFA compositions”) provide unprecedented potency in reducing AA plasma levels. The exceptional potency allows such n-3 FFA compositions to be used to treat, reverse, inhibit or prevent resistance to antiplatelet therapy in efficient converters using clinically relevant doses. The high potency also allows such n-3 FFA compositions to be administered at that same or at reduced dosage as an adjunct to antiplatelet therapy in patients who are not efficient converters—both those with elevated plasma AA levels and those with average AA plasma levels—with the potent AA-lowering effect of the n-3 FFA compositions improving the efficacy of antiplatelet therapies in nearly all such patients.

5.1. Determining “Efficient Converter” Status

Thus, in a first aspect, methods are provided herein for identifying subjects who are, or who will prove, resistant to antiplatelet therapy, such as therapy with clopidogrel or aspirin. The methods comprise determining, in a subject for whom antiplatelet therapy is clinically indicated, whether the subject is an efficient converter of mc-PUFAs to lc-PUFAs. Efficient converter status may be determined phenotypically, genotypically, or by combining phenotypic and genotypic determinations.

The term “polyunsaturated fatty acid” as used herein refers to a compound having the formula:

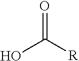

wherein R represents a C18 to C24 carbon chain with two or more double bonds. A mc-PUFA is a fatty acid that has a carbon chain (R) with up to 18 carbons. A lc-PUFA is a fatty acid that has a carbon chain (R) with 20 or more carbons. Polyunsaturated fatty acids can be denominated as “Ca:b”, wherein “a” is an integer that represents the total number of carbon atoms and “b” is an integer that refers to the number of double bonds in the carbon chain.

Two series of polyunsaturated fatty acids are relevant herein: omega-3 polyunsaturated fatty acids and omega-6 polyunsaturated fatty acids. The term “omega-3 fatty acid” or “omega-3 PUFA” as used herein refers to a polyunsaturated fatty acid wherein the first double bond is located after the third carbon in the carbon chain (R), numbering from the free methyl end of R. Omega-3 fatty acids may also be denominated “n-3” or “ω-3” fatty acids. The term “omega-6 fatty acid” as used herein refers to a polyunsaturated fatty acid wherein the first double bond is located after the sixth carbon in the carbon chain (R), counting from the free methyl end of R. Omega-6 fatty acids may also be referred to as “n-6” or “ω-6” fatty acids.

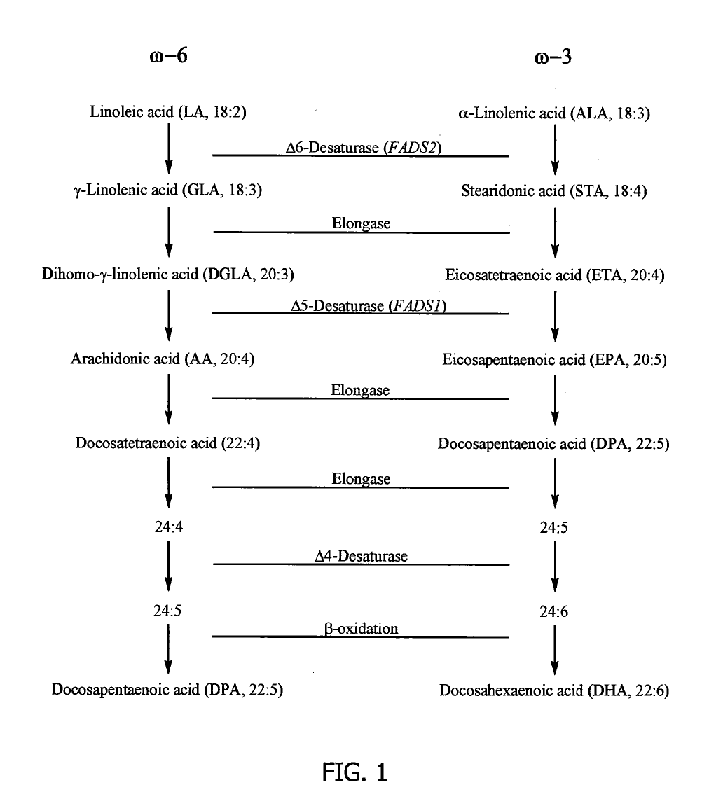

lc-PUFAs are obtained directly from the diet and are also synthesized metabolically from certain essential mc-PUFAs. With reference to FIG. 1, the medium chain C18:2 omega-6 fatty acid linoleic acid (“LA”) serves as a precursor for the synthesis of the C20:4 omega-6 arachidonic acid (“AA”), and the medium chain C18:3 omega-3 fatty acid α-linolenic acid (“ALA”) serves as the precursor for synthesis of the C20:5 omega-3 lc-PUFA eicosapentaenoic acid (“EPA”). As shown in FIG. 1, synthesis of the lc-PUFAs proceeds by elongation and desaturation steps catalyzed by specific elongase and desaturase enzymes.

As used herein, the term “efficient converter” refers to an individual who more efficiently synthesizes lc-PUFA products from mc-PUFAs precursors than an average individual. Efficient converter status can be determined phenotypically, by assessing one or more measures of efficiency of enzymatic conversion, genotypically, or by determining both phenotype and genotype.

5.1.1. Determining by Phenotype

As a consequence of increased enzymatic efficiency in the biosynthetic conversion of mc-PUFAs to lc-PUFAs, efficient converters have higher ratios of lc-PUFA products to respective mc-PUFA precursors (inversely, lower ratios of mc-PUFA precursors to respective lc-PUFA products), and will at times also have higher absolute levels of lc-PUFA products than individuals who are not efficient converters. Phenotypic determination of efficient converter status can thus be performed by determining and comparing the levels of mc-PUFA precursors to respective lc-PUFA products, by determining absolute levels of lc-PUFA products, by determining and comparing the levels of omega-6 and omega-3 lc-PUFAs, and/or by determining the omega-3 index (defined below). Because elongase and desaturase enzymes are shared by omega-6 and omega-3 fatty acid synthetic routes (see FIG. 1), phenotypic determination of efficient converter status can be performed by determining levels of omega-6 mc-PUFA precursors and their lc-PUFA products, omega-3 mc-PUFA precursors and their lc-PUFA products, or both. In typical embodiments, phenotypic determination is performed by measuring products and precursors in the omega-6 series.

The rate limiting enzymes in the conversion of dietary fatty acids to AA, EPA and other lc-PUFAs are the Δ5- and Δ6-fatty acid desaturases, which are respectively encoded by fatty acid desaturase (FADS) 1 and fatty acid desaturase (FADS) 2 genes on chromosome 11q12-13 in humans (see FIG. 1). In certain embodiments, therefore, the efficient converter phenotype is conferred by more efficient activity of one or both of the Δ5- and Δ6-fatty acid desaturases.

Accordingly, in certain embodiments, efficient converter status is usefully determined by determining and comparing the levels of products to precursors wherein at least one of Δ5- and Δ6-fatty acid desaturases is required for the synthetic conversion of the measured precursor to the measured product. In some embodiments, for example, status may be determined by measuring and comparing Δ5-fatty acid desaturase product AA and its immediate Δ5-fatty acid desaturase precursor, dihomo-γ-linolenic acid (C20:3 n-6) (“DGLA”). In certain embodiments, the lc-PUFA product AA is measured and compared to the levels of precursors earlier in the biosynthetic pathway, such as γ-linolenic acid (“GLA”) and/or linoleic acid (“LA”). In certain embodiments, efficient converter status may usefully be determined by measuring and comparing the levels of Δ6-desaturase fatty acid product GLA and its immediate Δ6-fatty acid desaturase precursor, LA.

Analogous determinations can be performed in the alternative or in addition in the omega-3 series. Thus, in some embodiments, the measured product:precursor ratio is the ratio of EPA to eicosatetraenoic acid (C20:4 n-3) (“ETA”). In some embodiments, the measured product:precursor ratio is the ratio of EPA to stearidonic acid (C18:4 n-3) (“STA”). In some embodiments, the measured product:precursor ratio is the ratio of EPA to α-linolenic acid (C18:3 n-3) (“ALA”). In some embodiments, the measured product:precursor ratio is the ratio of STA to ALA.

In certain embodiments, a subject is identified as an efficient converter if the product-to-precursor ratio is greater than 1. Accordingly, in some embodiments, a subject is determined to be an efficient converter if the subject's product: precursor ratio is at least about 1.5:1, at least about 2:1, at least about 2.5:1, at least about 3:1, at least about 3.5:1, at least about 4:1, at least about 4.5:1, at least about 5:1, at least about 5.5:1, at least about 6:1, at least about 6.5:1, at least about 7:1, at least about 7.5:1, at least about 8:1, at least about 8.5:1, at least about 9:1, at least about 9.5:1 at least about 10:1, at least about 11:1, at least about 12:1, at least about 13:1, at least about 14:1, or at least about 15:1. In certain embodiments, the subject is determined to be an efficient converter if the product:precursor ratio ranges between any the foregoing values, e.g., 2-6.5, 5-10, 6-8.5 or the like. In certain embodiments, a subject is identified as an efficient converter if the product:precursor ratio is at least about 6:1, at least about 6.5:1, at least about 7:1, at least about 7.5:1, at least about 8:1, at least about 8.5:1, at least about 9:1, at least about 9.5:1, at least about 10:1, at least about 11:1, at least about 12:1, at least about 12:1, at least about 13:1, at least about 14:1, or at least about 15:1.

In various embodiments, a subject is determined to be an efficient converter by measuring the fatty acid precursor-to-product ratio in the tissues of the efficient converter (“precursor:product ratio”). Thus, in some embodiments, the measured precursor:product ratio is the ratio of DGLA:AA. In other embodiments, the measured precursor:product ratio is the ratio of LA:GLA. In other embodiments, the measured precursor:product ratio is the ratio of LA:AA. In yet other embodiments, the measured precursor:product ratio is the ratio of GLA:AA. In various embodiments, the measured precursor:product ratio is the ratio of ETA:EPA. In some embodiments, the measured precursor:product ratio is the ratio of ALA:STA. In some embodiments, the measured precursor:product ratio is the ratio of ALA:EPA. In some embodiments, the measured precursor:product ratio is the ratio of STA:EPA.

In certain embodiments, a subject is identified as an efficient converter if the precursor:product ratio is less than 1. Accordingly, in some embodiments, a subject is determined to be an efficient converter if the precursor:product ratio is at least about 1:1.5, at least about 1:2, at least about 1:2.5, at least about 1:3, at least about 1:3.5, at least about 1:4, at least about 1:4.5, at least about 1:5, at least about 1:5.5, at least about 1:6, at least about 1:6.5, at least about 1:7, at least about 1:7.5, at least about 1:8, at least about 1:8.5, at least about 1:9, at least about 1:9.5, at least about 1:10, at least about 1:11, at least about 1:12, at least about 1:13, at least about 1:14, or at least about 1:15. In certain embodiments, the subject is determined to be an efficient converter if the precursor:product ratio ranges between any the foregoing values. In certain embodiments, a subject is identified as an efficient converter if the precursor:product ratio is at least about 1:6, at least about 1:6.5, at least about 1:7, at least about 1.7.5, at least about 1:8, at least about 1:8.5, at least about 1:9, at least about 1:9.5, at least about 1:10, at least about 1:11, at least about 1:12, at least about 1:13, at least about 1:14, or at least about 1:15.

In certain embodiments, a subject is determined to be an efficient converter by measuring the absolute levels of AA in one or more tissues of the subject, such as whole blood, red blood cells, plasma, or serum. In various embodiments, a subject is identified as an efficient converter if AA is present in one or more tissues in an amount that is greater than about 5%, greater than about 6%, greater than about 7%, greater than about 8%, greater than about 9%, greater than about 10%, greater than about 11%, greater than about 12%, greater than about 13%, greater than about 14% or greater than about 15% by weight of total fatty acids in the respective tissue. In various embodiments, a subject is determined to be an efficient converter if AA is present in the tissues in an amount of about 10% or more by weight of total fatty acids in the tissues.

Although there will be an increase in efficiency of conversion of mc-PUFAs to lc-PUFAs in both the omega-3 and omega-6 series, the efficient conversion phenotype will typically accentuate the difference in EPA and AA levels caused by differences in dietary consumption of omega-3 and omega-3 PUFAs and their respective precursors. Thus, in some embodiments, a subject is identified as an efficient converter by the EPA:AA ratio. In these embodiments, a subject is identified as an efficient converter if the EPA:AA ratio is less than about 1:10 (0.10). Thus, in certain embodiments, the subject is identified as an efficient converter if the EPA:AA ratio is less than about 1:15, less than about 1:20; and even lower.

In certain embodiments, a subject is identified as an efficient converter by the omega-3 index. As used herein, the term “omega-3 index” refers to the amount of EPA and DHA in a red blood cell sample expressed as a percent of total fatty acids in the red blood cell sample. Accordingly, in some embodiments, a subject is determined to be an efficient converter if the omega-3 index is less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 2%, less than about 1.5% or less than about 1% of total fatty acids. In particular embodiments, the subject is identified as an efficient converter if the omega-3 index is less than about 4% of total fatty acids. In certain embodiments, the subject is determined to be an efficient converter if the omega-3 index ranges between any the foregoing values.

Fatty acid levels can be measured in any bodily sample, including but not limited to, a sample of whole blood, plasma, serum, membranes of red blood cells, or adipose tissue. In some embodiments, the amount of a particular fatty acid is expressed as a percentage of the total fatty acids in the sample. Fatty acid levels can be measured by any method known in the art. In certain embodiments, fatty acid levels are measured by chromatographic methods, including but not limited to, gas chromatography, liquid chromatography-mass spectrometry, gas chromatography-mass spectrometry, and high performance liquid chromatography. In other embodiments, fatty acid levels are measured by spectroscopic methods, including but not limited to, nuclear magnetic resonance and Fourier transform infrared spectroscopy.

5.1.2. Determining by Genotype

Δ5- and Δ6-fatty acid desaturases are respectively encoded by fatty acid desaturase (FADS) 1 and fatty acid desaturase (FADS) 2 genes on chromosome 11q12-13 in humans (see FIG. 1). The term “fatty acid desaturase gene” or “FADS” as used herein refers to a gene encoding a fatty acid desaturase protein in a human or non-human animal that is necessary for the synthesis of lc-PUFAs. Fatty acid desaturase genes include the human FADS genes FADS1, which encodes the Δ5 desaturase (GenBank Accession No. NM_{—}013402.4), FADS 2, which encodes the Δ6-desaturase (GenBank Accession No. NM_{—}004265.2) and FADS 3 (GenBank Accession No. NM_{—}021727.3). Fatty acid desaturase genes and enzymes of non-human animals are readily ascertainable from GenBank (http://www.ncbi.nlm.nih.gov/genbank/).

Certain efficient converters have one or more polymorphisms in one or more fatty acid desaturase genes that lead to more efficient conversion of mc-PUFAs to lc-PUFAs. Referring to FIG. 1, in some embodiments the polymorphism is in the FADS2 gene, which encodes the Δ6-desaturase, and results in more efficient conversion of LA to γ-linolenic acid (“GLA”) and/or more efficient conversion of ALA to STA. In other embodiments, the polymorphism is in the FADS1 gene, which encodes the Δ5-desaturase, and results in more efficient conversion of DGLA to AA and/or more efficient conversion of eicosatetraenoic acid to EPA.

As used herein, a polymorphism “in a fatty acid desaturase gene” can be a polymorphism in the coding region of the gene, an intron, or in an upstream or downstream regulatory region of the gene. In various embodiments, the polymorphism is a single nucleotide polymorphism (“SNP”). Where the specific allelic variant is not specified herein, SNP refers to the minor variant (that is, the allele that has least prevalence in a population).

As discussed in further detail in Example 2, below, genotype at certain of these single nucleotide polymorphic sites correlates with higher baseline AA levels, and also correlates with increased responsiveness of AA plasma levels to treatment with a pharmaceutical composition comprising omega-3 PUFAs.

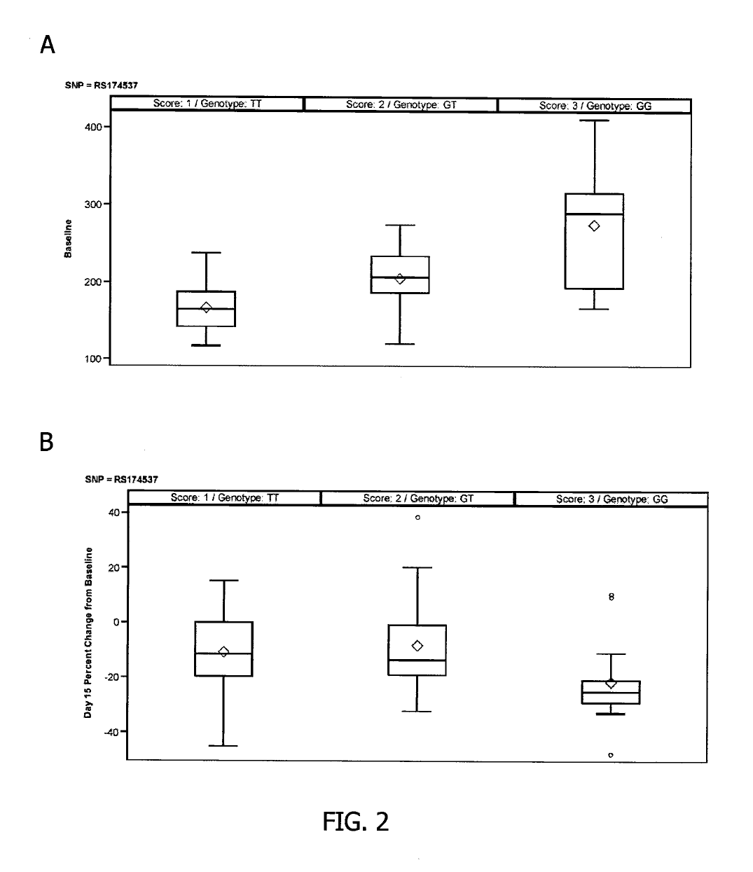

As shown in FIG. 2A, for example, subjects homozygous for the minor allele at

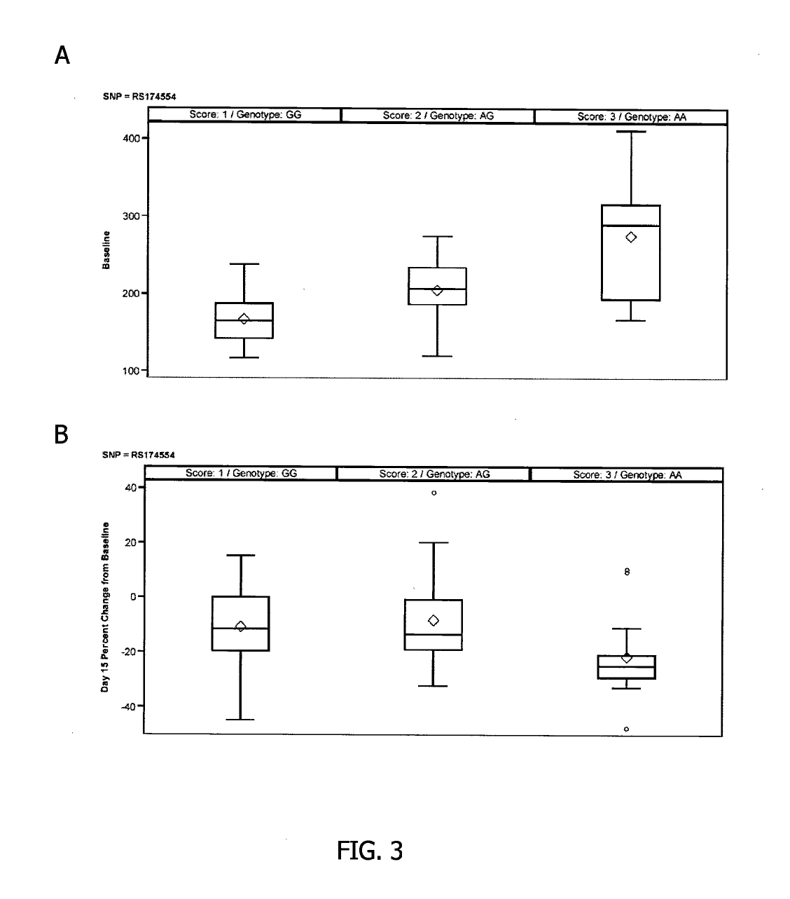

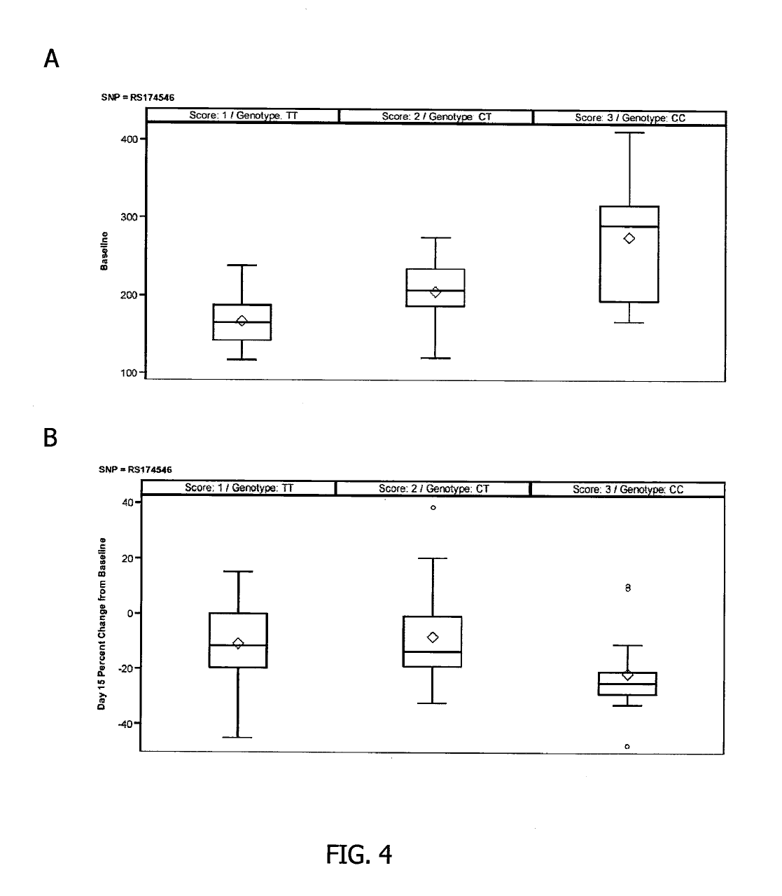

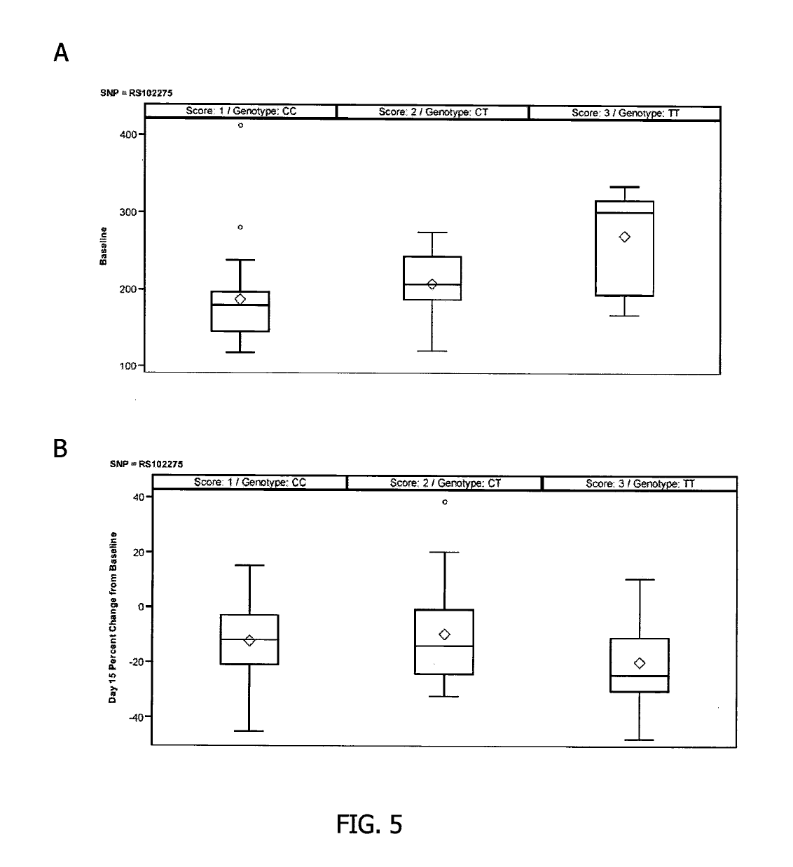

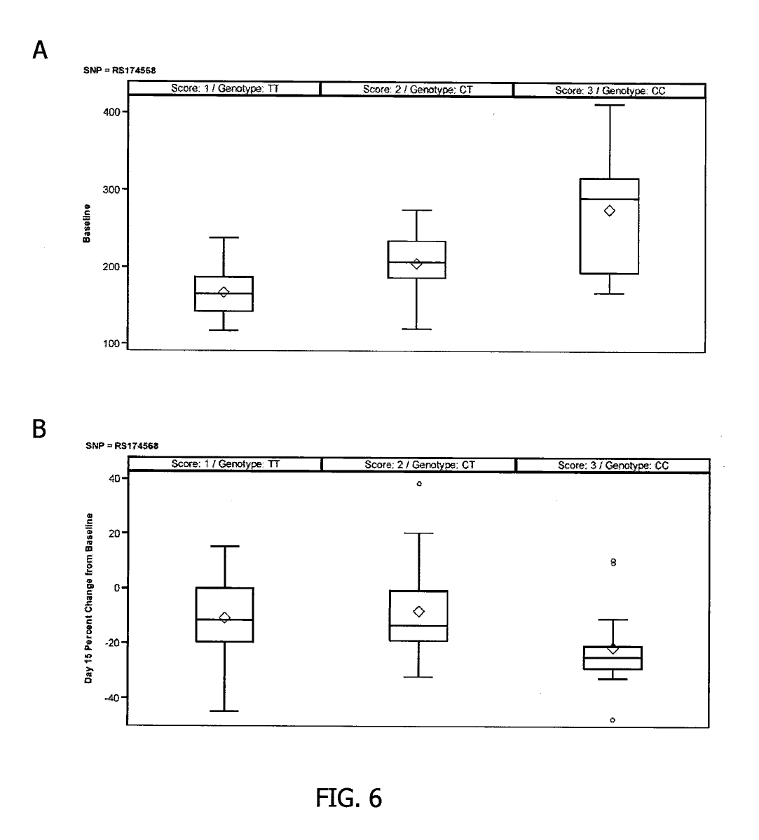

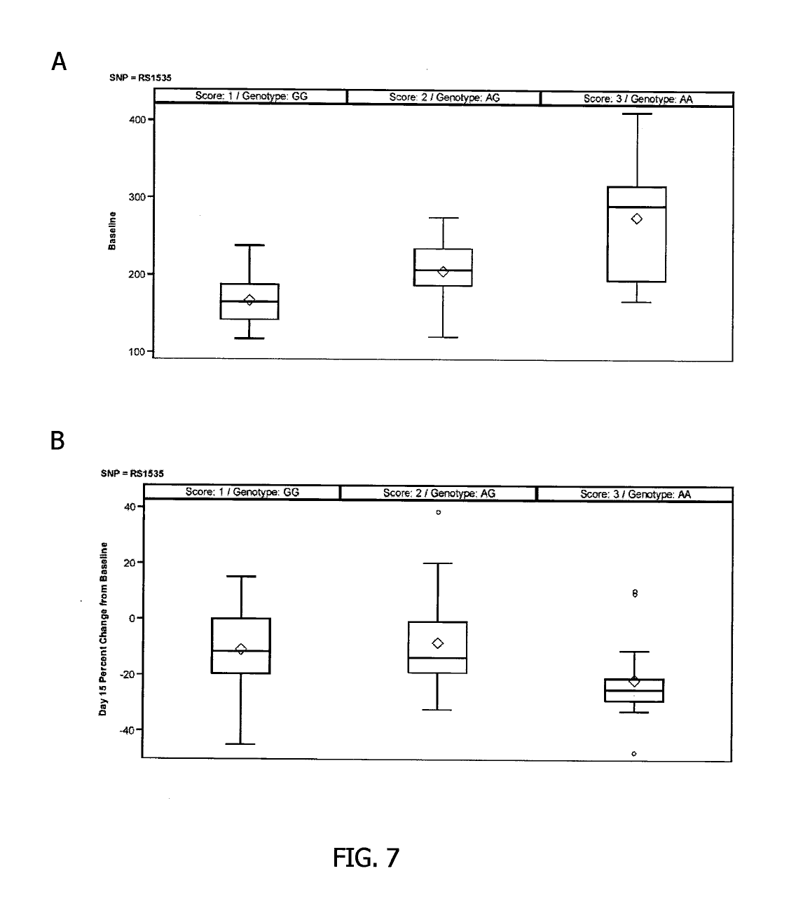

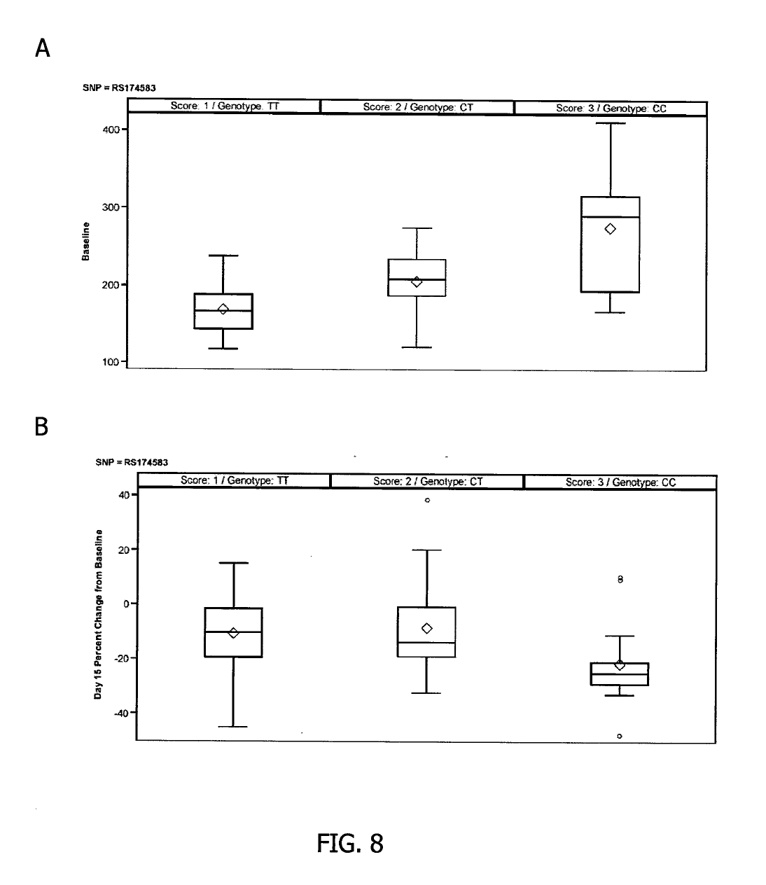

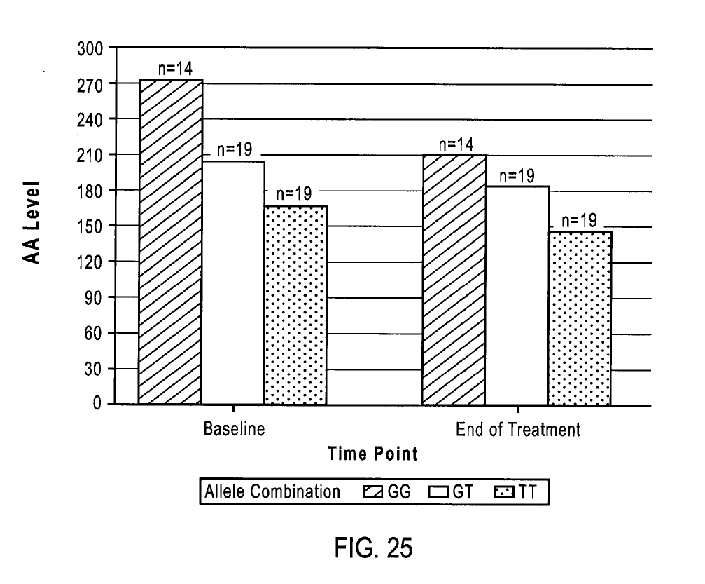

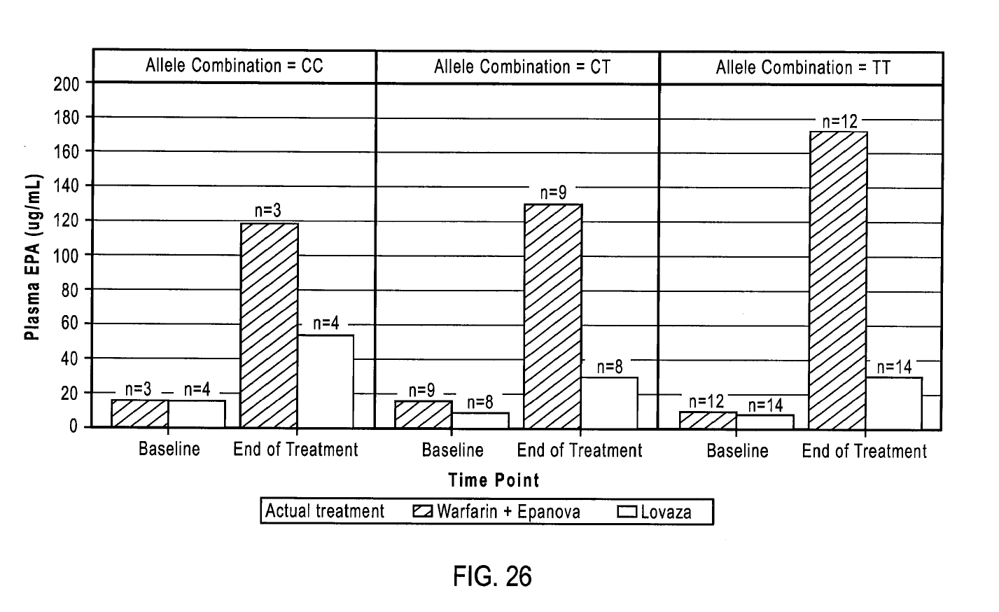

FADS1 SNP rs174537 (genotype: GG) have higher median and mean baseline AA levels, and therefore have greater potential resistance to anti-platelet therapies. As shown in FIG. 2B, these individuals have a greater percentage reduction in AA levels after two week treatment with an n-3 FFA composition compared to the other genotypes. Absolute baseline and EOT levels are shown in FIG. 25. Similar results were observed for FADS1 SNPs rs174554 (FIGS. 3A and 3B), rs174546 (FIGS. 4A and 4B), and rs102275 (FIGS. 5A and 5B). As further discussed in Example 2 and shown in FIG. 6A, subjects homozygous for the minor allele at FADS2 SNP rs174568 (genotype: CC) have higher median and mean baseline AA levels, and therefore have greater potential resistance to anti-platelet therapies. As shown in FIG. 6B, these individuals have a greater percentage reduction in AA levels with an n-3 FFA composition therapy compared to the other genotypes. Similar results are observed for FADS2 SNP rs1535 (FIGS. 7A and 7B) and rs174583 (FADS2 intronic) (FIGS. 8A and 8B).

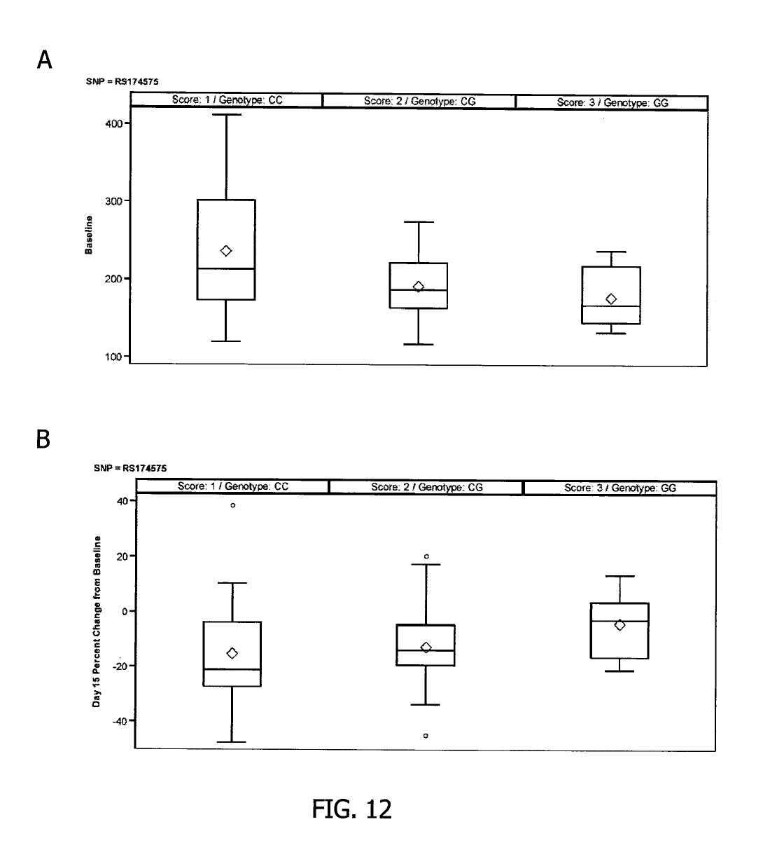

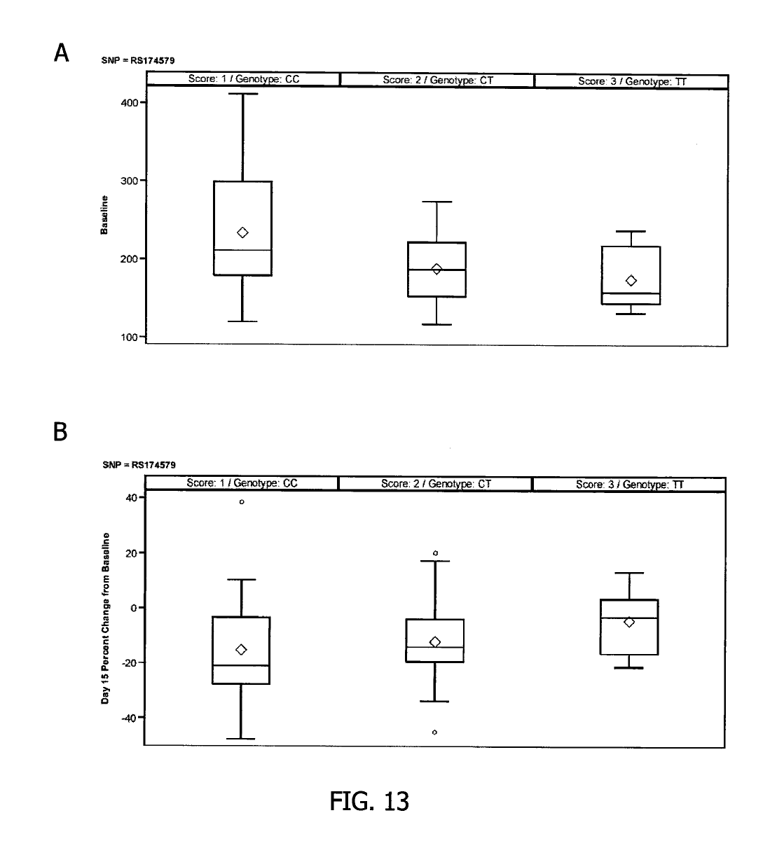

FIG. 12 (rs174575, FADS2) and FIG. 13 (rs174579, FADS2) demonstrate that for certain SNPs, subjects who are homozygous for the major allele, rather than minor allele, have higher average and median baseline AA levels. The plasma level of AA in individuals homozygous for the major allele at these two polymorphic sites is more responsive to 14-day treatment with an n-3 FFA composition than are the plasma levels for heterozygotes or those homozygous at the minor allele, those genotypes in which baseline plasma levels were lower.

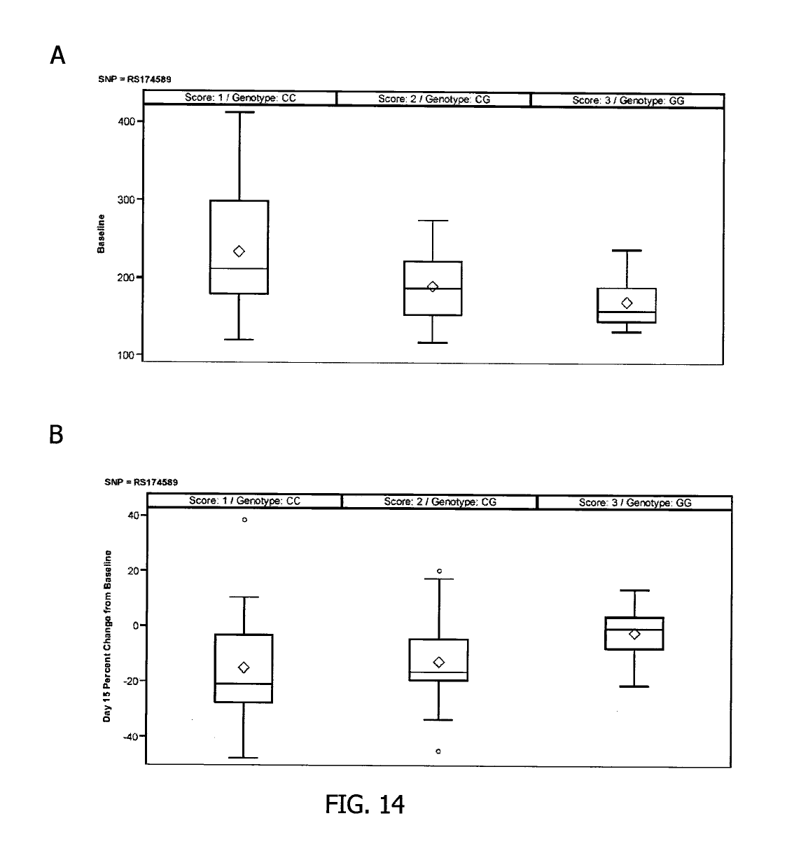

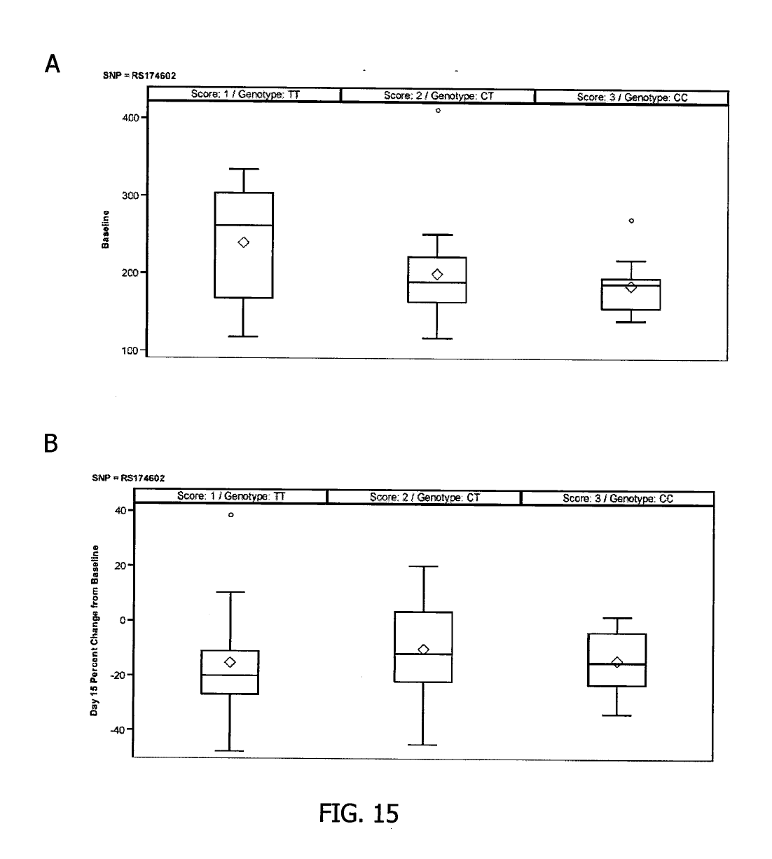

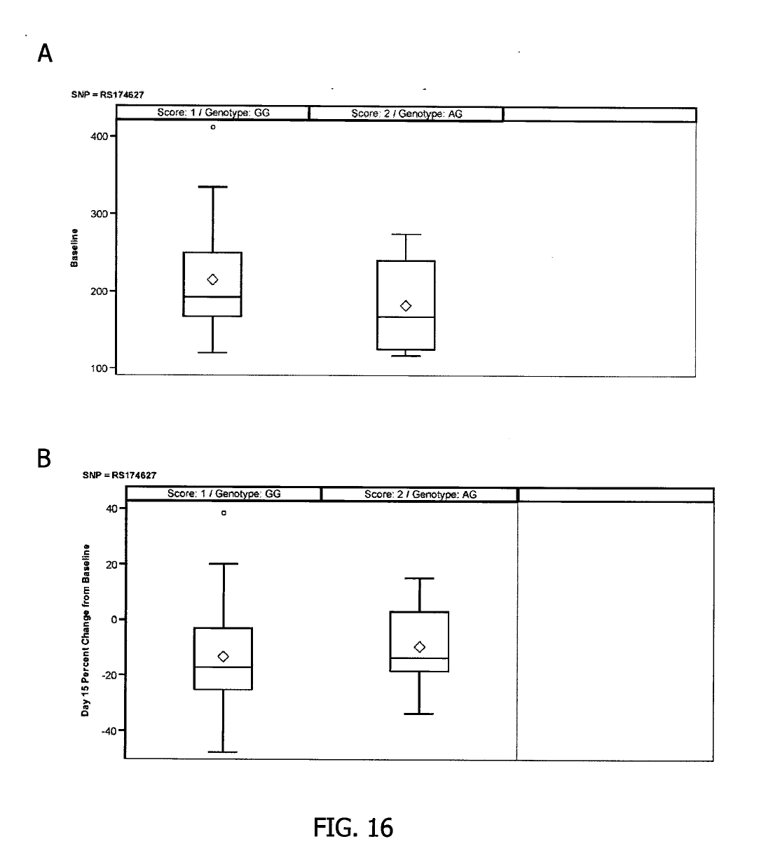

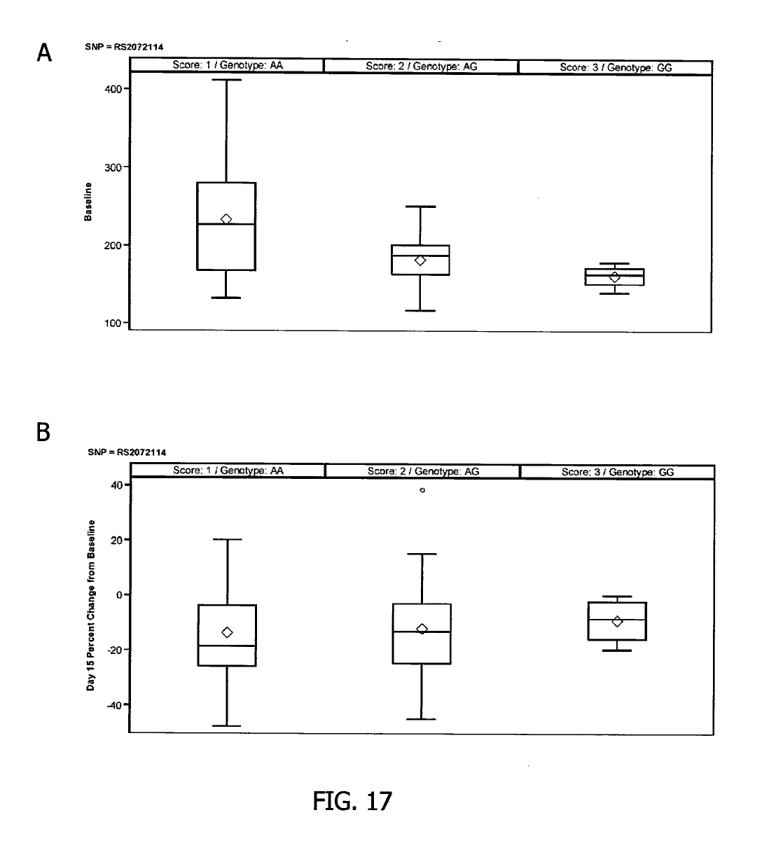

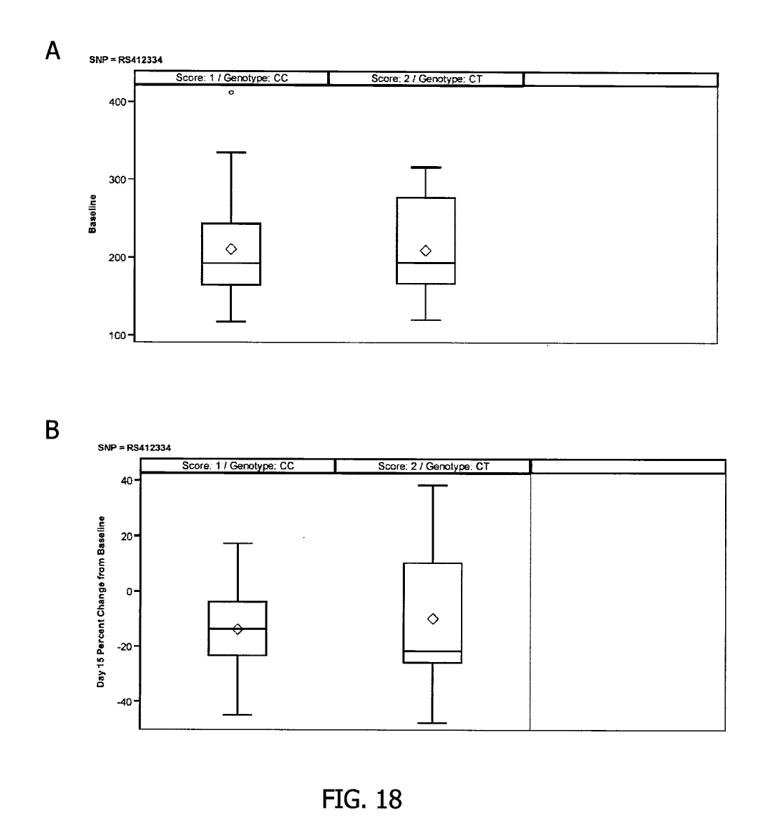

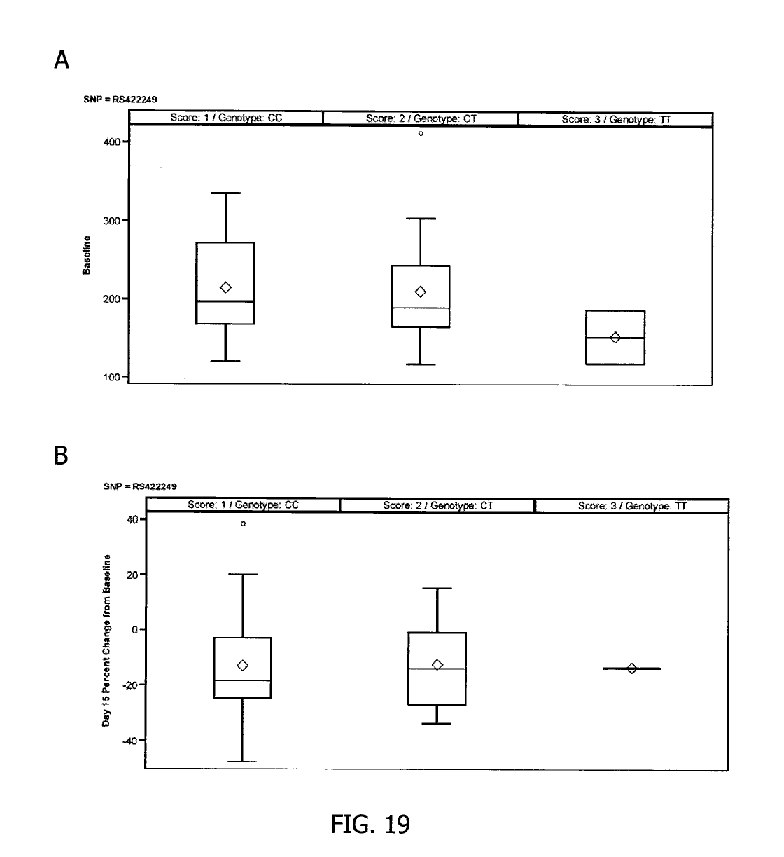

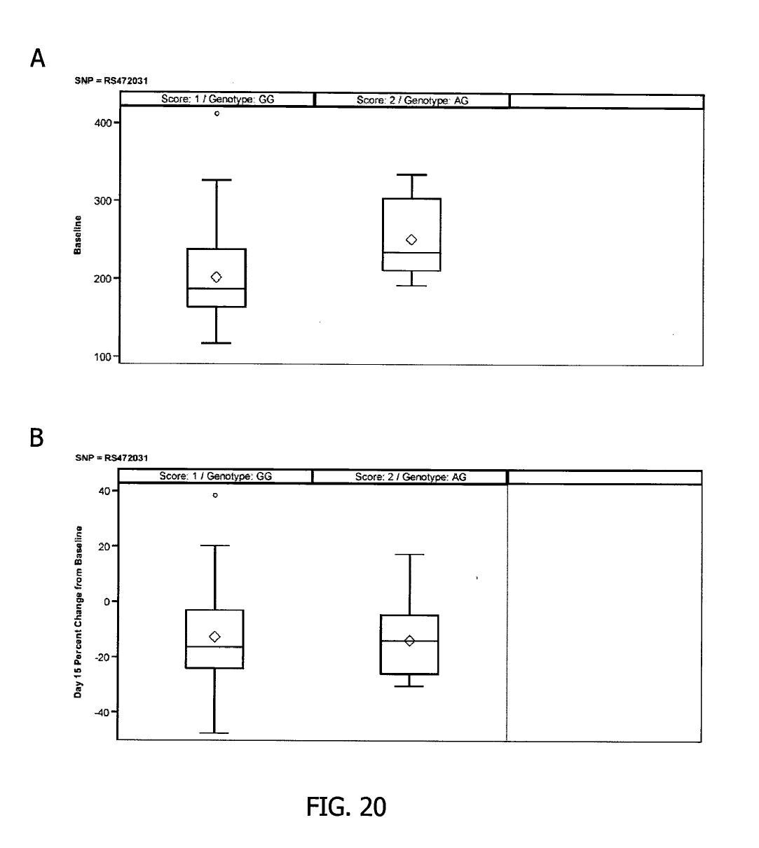

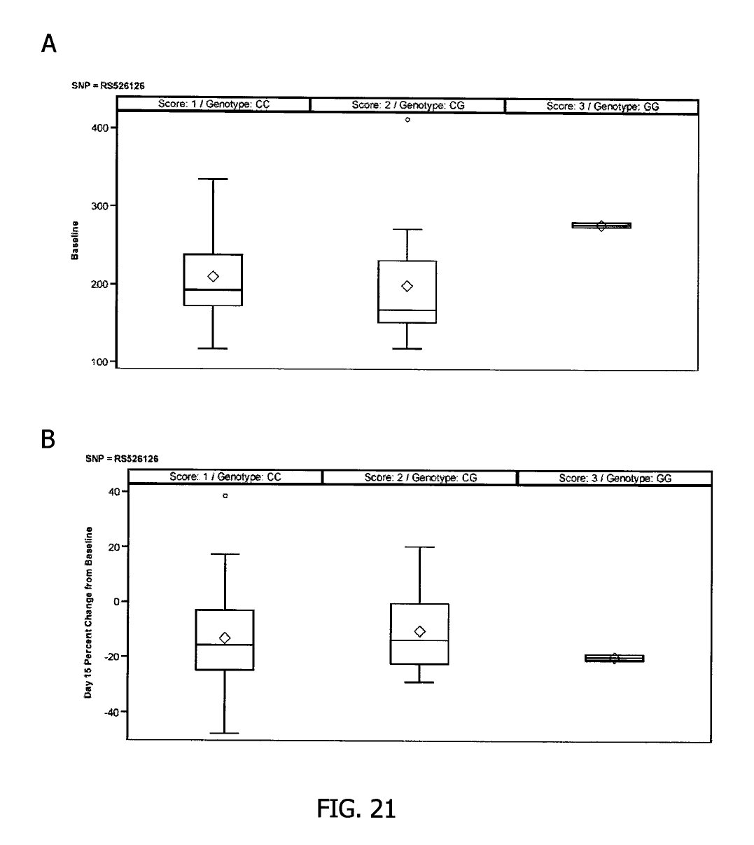

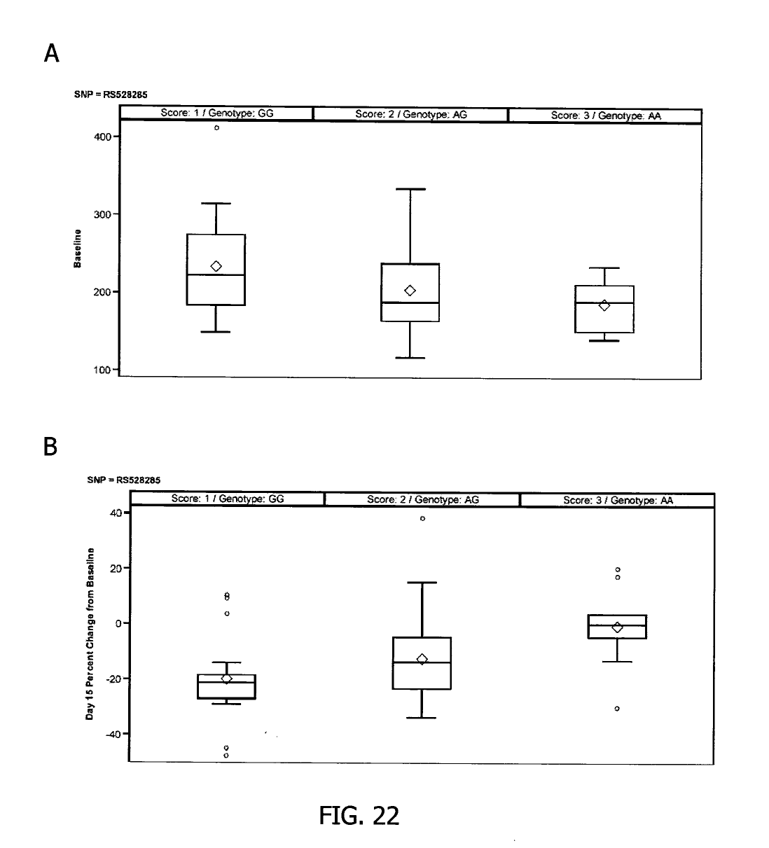

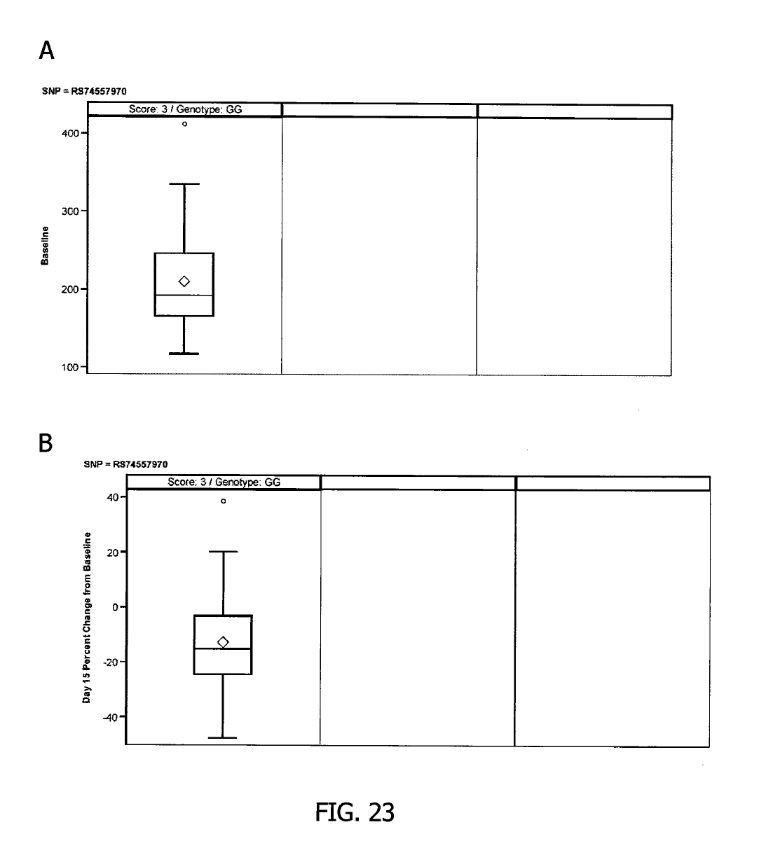

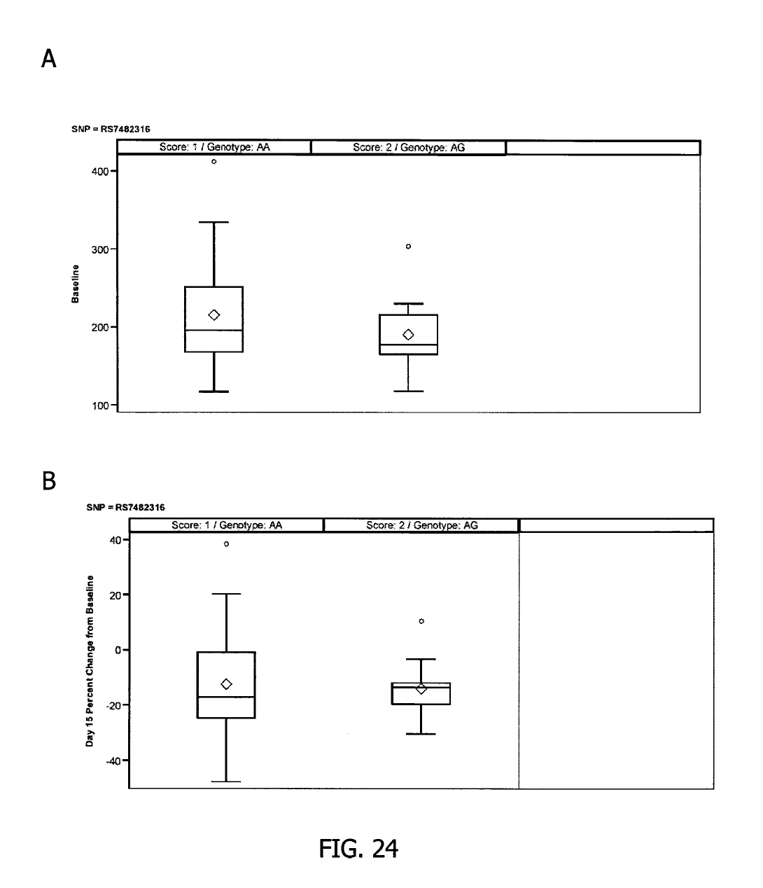

For other SNPs, genotypic contribution to baseline and post-treatment AA levels vary, as shown in FIGS. 14-24.

Accordingly, in certain embodiments, efficient converter status is usefully determined genotypically, for example by determining the presence of one or more polymorphisms associated with increased arachidonic acid levels at baseline. In various embodiments, efficient converter status is usefully determined by determining the presence of one or more polymorphisms associated with increased enzymatic efficiency of one or more desaturase enzymes in the biosynthetic pathway from mc-PUFAs to lc-PUFAs.

In certain embodiments, the allele at the polymorphic site that confers the efficient converter phenotype is the minor allele. In certain embodiments, the allele at the polymorphic site that confers the efficient converter phenotype is the major allele. In various embodiments, the polymorphism is a SNP in the FADS1 gene, such as rs174537, rs174554, rs174546, or rs102275. In some embodiments, the polymorphism is a SNP in the FADS2 gene, such as rs174568 or rs1535. In some embodiments, the polymorphism is a SNP such as rs174556, rs174549, rs174555, rs174556, rs174576, rs174579, rs968567, rs173534, rs174567, or those identified in FIGS. 2-24. Other single nucleotide polymorphisms found in FADS1 and FADS2 genes of humans and non-human animals can be found in the NCBI SNP database “dbSNP”, available at http://www.ncbi.nlm.nih.gov/projects/SNP/.

Polymorphisms, including single nucleotide polymorphisms, can be detected in a sample, e.g., a sample containing nucleated blood cells, by any method known in the art. Methods of detecting SNPs include DNA sequencing, methods that require allele specific hybridization of primers or probes (e.g., dynamic allele-specific hybridization (DASH)), use of molecular beacons, and SNP microarrays such as the Affymetrix Human SNP Array 6.0), allele-specific incorporation of nucleotides to primers bound close to or adjacent to the polymorphisms (“single base extension”, or “minisequencing”), allele-specific ligation of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation may also be employed where the SNP lies in a coding region and results in an amino acid change.

5.1.3. Determining by Both Phenotype and Genotype

In certain embodiments, a subject is identified as an efficient converter by both phenotypic and genotypic determination, as above described.

In certain embodiments, for example, a subject is identified as an efficient converter by determining a ratio of AA:DGLA and by detecting an efficient converter allele at a single nucleotide polymorphism site selected from rs174537, rs174554, rs174546, rs102275, rs174568, rs1535, and rs174583, or combinations thereof. In some embodiments, a subject is identified as an efficient converter by determining a ratio of AA:DGLA and by detecting an efficient converter allele by genotyping at a single nucleotide polymorphism site selected from rs174537, rs102275, rs174546, rs174556, rs1535, rs174576, rs174579, rs968567, rs173534, rs174549, rs174555, rs174556, rs174568, rs174567 and combinations thereof. In particular embodiments, the AA:DGLA ratio is greater than about 6.

In certain embodiments, a subject is identified as an efficient converter by determining the absolute level of AA in a body tissue of the subject and by detecting an efficient converter allele at a single nucleotide polymorphism site selected from rs174537, rs174554, rs174546, rs102275, rs174568, rs1535, and rs174583, or combinations thereof. In some embodiments, a subject is identified as an efficient converter by determining the level of AA in a body tissue and by detecting an efficient converter allele at a single nucleotide polymorphism site in a fatty acid desaturase gene selected from rs174537, rs102275, rs174546, rs174556, rs1535, rs174576, rs174579, rs968567, rs173534, rs174549, rs174555, rs174556, rs174568, rs174567 and combinations thereof. In particular embodiments, the AA level is greater than about 10% by weight of total fatty acids in the sample

In other embodiments, a subject is identified as an efficient converter by determining a ratio of EPA:AA and by detecting the presence of an efficient converter allele at a SNP site selected from rs174537, rs174554, rs174546, rs102275, rs174568, rs1535, and rs174583, or combinations thereof. In some embodiments, a subject is identified as an efficient converter by determining the EPA:AA ratio and by detecting an efficient converter allele at a single nucleotide polymorphism site selected from rs174537, rs102275, rs174546, rs174556, rs1535, rs174576, rs174579, rs968567, rs173534, rs174549, rs174555, rs174556, rs174568, rs174567 and combinations thereof. In certain embodiments, the EPA:AA ratio is less than about 0.10.

In further embodiments, a subject is identified as an efficient converter by determining the omega-3 index and by detecting the presence of an efficient converter allele at a single nucleotide polymorphic site selected from rs174537, rs174554, rs174546, rs102275, rs174568, rs1535, and rs174583, or combinations thereof. In some embodiments, a subject is identified as an efficient converter by determining the omega-3 index and by detecting an efficient converter allele by genotyping at a single nucleotide polymorphism site gene selected from rs174537, rs102275, rs174546, rs174556, rs1535, rs174576, rs174579, rs968567, rs173534, rs174549, rs174555, rs174556, rs174568, rs174567 and combinations thereof. In particular embodiments, the subject is identified as an efficient converter if the omega-3 index is less than about 4% of total fatty acids.

5.2. Methods of Treatment

5.2.1. Methods of Treating, Reversing, Inhibiting, or Preventing Resistance to Antiplatelet Therapy in Efficient Converters

As noted above and discussed in detail in Example 2 below, and as further illustrated in FIGS. 2-25, subjects who have genotypes that are correlated with elevated plasma arachidonic acid levels also have increased responsiveness of plasma arachidonic acid levels to treatment with a pharmaceutical composition comprising omega-3 PUFAs.

Accordingly, in another aspect, methods are provided herein for treating, reversing, inhibiting, or preventing resistance to antiplatelet therapy in a subject who is an efficient converter of mc-PUFAs to lc-PUFAs and for whom antiplatelet therapy is clinically indicated. The methods comprise administering to a subject who has been determined to be an efficient converter of mc-PUFAs to lc-PUFAs, and for whom antiplatelet therapy is clinically indicated, an amount of a composition comprising omega-3 lc-PUFAs (“omega-3 composition”) effective to treat, reverse, inhibit or prevent resistance to antiplatelet therapy.

In typical embodiments, subjects are determined to be efficient converters according to the methods above-described. In certain embodiments, the methods further include determining whether the subject has a poor clopidogrel metabolizer genotype. As used herein, a subject who has a “poor clopidogrel metabolizer genotype” refers to a subject who has a polymorphism in a gene encoding a cytochrome P450 isozyme gene that confers a poor metabolizer phenotype. In certain embodiments, the gene encodes a cytochrome P450 isozyme selected from CYP2C19, CYP3A5, CYP2C9 and CYP2B6. In particular embodiments, the polymorphism is a single nucleotide polymorphism selected from the group consisting of rs4244285, rs4986893, rs28399504, rs12248560, rs776746, rs1057910, rs3745274 and combinations thereof.

Compositions comprising omega-3 lc PUFAs suitable for use in the methods are described in Section 5.3, below. Effective amounts for use are described in Section 5.2.4 below, and dosage schedules are described in Section 5.2.5 below.

5.2.2. Methods of Treating Efficient Converters with Antiplatelet Therapy

In another aspect, methods are provided for providing antiplatelet therapy to subjects in need thereof, comprising (a) determining whether the subject is an efficient converter of mc-PUFA to lc-PUFA and (b) in those subjects determined to be efficient converters of mc-PUFA to lc-PUFA, adjunctively administering (i) an amount of a composition comprising omega-3 lc-PUFAs effective to treat, reverse, inhibit, or prevent resistance to antiplatelet therapy, and (ii) an effective amount of an antiplatelet agent.

In a further aspect, improved methods of providing antiplatelet therapy to subjects in need thereof are provided. The improvement comprises determining whether the subject is an efficient converter of mc-PUFA to lc-PUFA and in those subjects determined to be efficient converters of mc-PUFA to lc-PUFA, adjunctively administering an amount of a composition comprising omega-3 lc-PUFAs effective to treat, reverse, inhibit, or prevent resistance to antiplatelet therapy.

In typical embodiments, subjects are determined to be efficient converters according to the methods above-described. In certain embodiments, the methods further include determining whether the subject has a poor clopidogrel metabolizer genotype. As used herein, a subject who has a “poor clopidogrel metabolizer genotype” refers to a subject who has a polymorphism in a gene encoding a cytochrome P450 isozyme gene that confers a poor metabolizer phenotype. In certain embodiments, the gene encodes a cytochrome P450 isozyme selected from CYP2C19, CYP3A5, CYP2C9 and CYP2B6. In particular embodiments, the polymorphism is a single nucleotide polymorphism selected from the group consisting of rs4244285, rs4986893, rs28399504, rs12248560, rs776746, rs1057910, rs3745274 and combinations thereof.

Compositions comprising omega-3 lc-PUFAs suitable for use in the methods are described in Section 5.3, below. In certain embodiments, the composition comprising omega-3 lc-PUFAs is included in a dual dosage form of the type described in Section 5.3.4. Effective amounts of compositions comprising lc-omega-3 PUFAs and antiplatelet agents are described in Section 5.2.4 below, and dosage schedules are described in Section 5.2.5 below.

The composition comprising omega-3 lc-PUFAs is administered concomitantly with or adjunctively with an effective amount of antiplatelet therapy. By “concomitant” or “adjunctive” administration, it is intended that the composition comprising omega-3 lc-PUFAs be administered at and for a time sufficient to ensure reduction in plasma AA levels concurrently with the presence in the blood of the antiplatelet agent. Thus, the omega-3 lc-PUFA composition can be administered concurrently with administration of antiplatelet therapy, and may be started before, and/or continued after, cessation of antiplatelet therapy.

The terms “antiplatelet therapy” or “platelet inhibitor therapy” as used herein refer to administration of one or more agents that interfere with the ability of platelets to aggregate.

Agents useful for antiplatelet therapy (“antiplatelet agents”) are known. In certain embodiments, the antiplatelet therapy agent is an adenosine diphosphate (ADP) receptor inhibitor, such as clopidogrel (Plavix®), ticlopidine (Ticlid®), prasugrel (Effient®), and ticagrelor (Brilinta®); phosphodiesterase inhibitors such as cilostazol (Pletal®); glycoprotein IIb/IIIa inhibitors such as abciximab (ReoPro®), eptifibatide (Integrilin®) and tirofiban (Aggrastat®). In various embodiments, the antiplatelet therapy agent is an adenosine reuptake inhibitor such as dipyridamole (Persantine®); a thromboxane inhibitor, e.g., thromboxane synthase inhibitors or thromboxane receptor antagonists such as terutroban, and combinations thereof.

In certain embodiments, the antiplatelet therapy agent is a non-steroidal anti-inflammatory drug selected from the group consisting of aspirin, aloxiprin, benorylate, diflunisal, ethenzamide, magnesium salicylate, methyl salicylate, salsalate, salicin, salicylamide, sodium salicylate, arylalkanoic acids, diclofenac, aceclofenac, acemetacin, alclofenac, bromfenac, etodolac, indometacin, indometacin farnesil, mabumetone, oxametacin, proglumetacin, sulindac, tolmetin, ibuprofen, alminoprofen, benoxaprofen, carprofen, dexibuprofen, dexketoprofen, fenbufen, fenoprofen, flunoxaprofen, flurbiprofen. ibuproxam, indoprofen, ketoprofen, ketorolac, loxoprofen, miroprofen, naproxen, oxaprozin, pirprofen, suprofen, tarenflurbil, tiaprofenic acid, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, sulfinpyrazone, piroxicam, droxicam, lornoxicam, meloxicam, tenoxicamm ampiroxicam, celecoxib, deracoxib, etoricoxib, firocoxib, lumiracoxib, parecoxib, rofecoxib, valdecoxib, nimesulide, naproxcinod, fluproquazone, and combinations thereof.

In particular embodiments, the antiplatelet agent is clopidogrel. In some embodiments, the antiplatelet agent is aspirin. In certain embodiments, the antiplatelet agent is a combination of clopidogrel and aspirin.

A subject who is being treated with an antiplatelet therapy, or for whom an antiplatelet therapy is clinically indicated, may suffer from one or more clinical indications that results in blood vessel damage.

Thus, in certain embodiments, the methods comprise (a) determining whether a subject suffering from one or more clinical indications that results in blood vessel damage is an efficient converter of mc-PUFA to lc-PUFA and (b) in those subjects determined to be efficient converters of mc-PUFA to lc-PUFA, adjunctively administering (i) an amount of a composition comprising omega-3 lc-PUFAs effective to treat, reverse, inhibit, or prevent resistance to antiplatelet therapy, and (ii) an effective amount of an antiplatelet agent.

In some embodiments, for example, the method comprises determining whether a subject has a clinical indication selected from the group consisting of acute coronary syndromes (“ACS”), including non-ST-segment elevation ACS (unstable angina/non-ST-elevation myocardial infarction (NSTEMI)), and acute ST segment elevation myocardial infarction (STEMI), arteriosclerotic vascular disease, myocardial infarction (MI), cerebrovascular accident, e.g., recent stroke, and established peripheral occlusive arterial disease.

In certain embodiments, the method comprises determining whether a subject has a clinical indication selected from the group consisting of transient ischemia of the brain, coronary angioplasty, stent implantation, lower limb arterial graft, carotid endoarterectomy, coronary artery bypass, atrial fibrillation, postoperative thromboembolic complications of cardiac valve replacement, thrombocythemia secondary to myeloproliferative disorders and intermittent claudication. In other embodiments, the subject has a high risk of developing cardiovascular disease.

5.2.3. Improved Methods of Inhibiting Platelet Aggregation

As further described in Example 3, the inventors have discovered that compositions comprising lc-omega-3 PUFAs in free acid form (“n-3 FFA compositions”) provide unprecedented potency in reducing AA plasma levels. The exceptional potency allows such n-3 FFA compositions to be used not only to treat, reverse, inhibit, or prevent resistance to antiplatelet therapy in efficient converters, but additionally allow's such n-3 FFA compositions to be administered at that same or at reduced dosages as an adjunct to antiplatelet therapy in patients who are not efficient converters—including those with elevated plasma AA levels and those with average AA plasma levels—with the potent AA-lowering effect of the n-3 FFA compositions improving the efficacy of antiplatelet therapies in nearly all such patients.

Thus, in another aspect, improved methods of treating a patient with an antiplatelet agent are provided, wherein the improvement comprises adjunctively administering an n-3 FFA composition in an amount effective to improve the efficacy of the antiplatelet therapy. In a related aspect, methods of treating a patient with an antiplatelet agent are provided. The methods comprise (a) administering a therapeutically effective amount of an inhibitor of platelet aggregation; and (b) adjunctively administering an n-3 FFA composition in an amount effective to improve the efficacy of the antiplatelet therapy.

n-3 FFA compositions suitable for use in the methods described in this subsection are described in Section 5.3.2. In certain embodiments, the n-3 FFA composition is included in a dual dosage form of the type described in Section 5.3.4. Effective amounts of n-3 FFA and antiplatelet agent are described in Section 5.2.4. n-3 FFA composition dosage schedules are described in Section 5.2.5.

The n-3 FFA composition is administered concomitantly with or adjunctively with an effective amount of antiplatelet therapy. In certain embodiments, the antiplatelet therapy agent is an adenosine diphosphate (ADP) receptor inhibitor, such as clopidogrel (Plavix®), ticlopidine (Ticlid®), prasugrel (Effient®), and ticagrelor (Brilinta®); phosphodiesterase inhibitors such as cilostazol (Pletal®); glycoprotein IIb/IIIa inhibitors such as abciximab (ReoPro®), eptifibatide (Integrilin®) and tirofiban (Aggrastat®). In various embodiments, the antiplatelet therapy agent is an adenosine reuptake inhibitor such as dipyridamole (Persantine®); a thromboxane inhibitor, e.g., thromboxane synthase inhibitors or thromboxane receptor antagonists such as terutroban, and combinations thereof.

In certain embodiments, the antiplatelet therapy agent is a non-steroidal anti-inflammatory drug selected from the group consisting of aspirin, aloxiprin, benorylate, diflunisal, ethenzamide, magnesium salicylate, methyl salicylate, salsalate, salicin, salicylamide, sodium salicylate, arylalkanoic acids, diclofenac, aceclofenac, acemetacin, alclofenac, bromfenac, etodolac, indometacin, indometacin farnesil, mabumetone, oxametacin, proglumetacin, sulindac, tolmetin, ibuprofen, alminoprofen, benoxaprofen, carprofen, dexibuprofen, dexketoprofen, fenbufen, fenoprofen, flunoxaprofen, flurbiprofen. ibuproxam, indoprofen, ketoprofen, ketorolac, loxoprofen, miroprofen, naproxen, oxaprozin, pirprofen, suprofen, tarenflurbil, tiaprofenic acid, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, sulfinpyrazone, piroxicam, droxicam, lornoxicam, meloxicam, tenoxicamm ampiroxicam, celecoxib, deracoxib, etoricoxib, firocoxib, lumiracoxib, parecoxib, rofecoxib, valdecoxib, nimesulide, naproxcinod, fluproquazone, and combinations thereof.

In particular embodiments, the antiplatelet agent is clopidogrel. In some embodiments, the antiplatelet agent is aspirin. In certain embodiments, the antiplatelet agent is a combination of clopidogrel and aspirin.

In certain embodiments, an amount of n-3 FFA composition is administered effective to reduce plasma arachidonic acid levels by at least 5% (as compared to levels prior to administration). In some embodiments, the amount is effective to reduce plasma AA levels by at least 10%, 15%, even at least 20%, 25% or more. In certain embodiments, the amount of composition comprising omega-3 lc-PUFAs is sufficient to reduce plasma AA to values that are average among individuals who are not resistant to antiplatelet therapy. In various embodiments, an amount of n-3 FFA composition is administered effective to reduce plasma arachidonic acid levels by at least 25 μg/mL, by at least 50 μg/mL, by at least 75 μg/mL, even by at least 100 μg/mL. In various embodiments, the n-3 FFA composition is administered in an amount sufficient to provide an EPA/AA ratio of at least about 0.30, at least about 0.40, at least about 0.50, at least about 0.60, at least about 0.65, even at least about 0.70.

5.2.4. Effective Amounts

The methods described herein comprise administering an amount of a composition comprising omega-3 lc-PUFAs that is effective to treat, reverse, inhibit, or prevent resistance to antiplatelet therapy.

In various embodiments, the effective amount is prior-determined.

In other embodiments, the methods further comprise titrating dosage to achieve a desired degree of efficacy of antiplatelet therapy. Optionally, the methods further comprise adjusting dosage of the composition comprising omega-3 lc-PUFAs after measuring the efficacy of antiplatelet therapy.

The effect of an antiplatelet therapy on platelet function can be measured by any method known in the art.

In certain embodiments, platelet function can be measured by non-biochemical methods e.g., light transmittance or electrical impedance, to measure platelet aggregation. In a particular embodiment, resistance is measured by measuring adenosine diphosphate (ADP)-induced platelet aggregation by LTA, as measured before and after taking the antiplatelet agent. “LTA” is optical light transmission aggregometry in which the decreased turbidity of a platelet-rich plasma sample in which platelets are aggregating is measured by an increase in light transmission over time. In certain embodiments, a decrease in platelet aggregation of greater than or equal to 30% is evidence of the antiplatelet agent's effectiveness. In other embodiments, resistance is from less than 10% decrease in ADP-induced aggregation by LTA to less than 20% decrease in aggregation.

In other embodiments, platelet function is measured by biochemical methods. Examples of biochemical assays include, but are not limited to, measuring serum or urine thromboxane B2 using, e.g., an ELISA assay, and measuring the vasodilator-stimulated phosphoprotein platelet reactivity index (VASP-PRI) using, e.g., flow cytometry. Commercially available point-of-care platelet function devices can also be used to measure platelet response in a subject. Point-of-care devices include the Platelet Function Analyzer-100 (PFA-100, Dade-Behring, Miami, Fla.), which measures platelet function under high shear stress by drawing blood through a small aperture and measuring how quickly the aperture is “closed” by a platelet plug, the VerifyNow™ assay (Accumetrics, San Diego, Calif.), which uses light-based whole blood aggregometry, and the Thromboelastograph (TEG™), which measures clot tensile strength.

In some embodiments, an effective amount of an omega-3 lc-PUFA composition described herein is one that reduces the vasodilator-stimulated phosphoprotein platelet reactivity index (VASP-PRI) from greater than about 50% to from about 40% to about 50%. In other embodiments, an effective amount of an omega-3 fatty acid composition is one that achieves less than about 236 P2Y12 receptor reaction units as measured by response to adenosine diphosphate (ADP) in a VerifyNow P2Y12 assay. In still other embodiments, an effective amount of an omega-3 lc-PUFA composition is one that induces less than about 46% maximal 5 μM ADP-induced platelet aggregation, preferably from about 46% to about 40% maximal 5 μM ADP-induced platelet aggregation, as measured by turbidometry in platelet-rich plasma. In further embodiments, an effective amount of an omega-3 lc-PUFA composition is one that achieves less than about 468 arbitrary aggregation units per minute, preferably between about 188 and about 468 arbitrary aggregation units per minute, in response to ADP, as measured by Multiplate® impedance aggregometry. In various embodiments, an effective amount of an omega-lc-PUFA composition for treating or preventing resistance to an antiplatelet therapy achieves desirable endpoints in two or more of the P2Y12 assay, the ADP-induced platelet aggregation assay, and arbitrary aggregation units per minute in response to ADP assay. In a particular embodiment, an effective amount of an omega-3 lc-PUFA composition achieves desirable endpoints in all three assays.

In certain embodiments, the methods further comprise titrating dosage of the composition comprising omega-3 lc-PUFAs to achieve a desired absolute plasma level of AA; a desired percentage degree of reduction in plasma level of AA; a desired absolute or percentage reduction in blood or serum levels of AA; a desired EPA/AA ratio; and/or a desired omega-3 index. Optionally, the methods further comprise adjusting dosage of the composition comprising omega-3 lc-PUFAs after measuring PUFA levels.

In typical embodiments, the amount of composition comprising omega-3 lc-PUFAs that is effective to treat, reverse, inhibit or prevent resistance to antiplatelet therapy is an amount that is effective to reduce plasma arachidonic acid levels by at least 5%. In some embodiments, the amount is effective to reduce plasma AA levels by at least 10%, 15%, even at least 20%, 25% or more. In certain embodiments, the amount of composition comprising omega-3 lc-PUFAs is sufficient to reduce plasma AA to values that are average among individuals who are not resistant to antiplatelet therapy.

In various embodiments, the amount of composition comprising omega-3 lc-PUFAs is effective to reduce plasma arachidonic acid levels by at least 25 μg/mL, by at least 50 μg/mL, by at least 75 μg/mL, even by at least 100 μg/mL.

In various embodiments, the amount of composition comprising omega-3 lc-PUFAs is effective to product an EPA/AA ratio of at least about 0.30, at least about 0.40, at least about 0.50, at least about 0.60, at least about 0.65, even at least about 0.70.

Levels of lc-PUFAs in the body of the subject can be ascertained by any method known in the art. Exemplary methods of monitoring lc-PUFA levels in a biological sample include, but are not limited to, chromatographic methods such as gas chromatography (GC), gas liquid chromatography (GLC), mass spectrometry (MS), high performance liquid chromatography (HPLC), reverse phase HPLC, thin layer chromatography (TLC), GC-MS and TLC-GLC, and the like, and spectroscopic methods such as nuclear magnetic resonance spectroscopy (NMR) and Fourier transform infrared spectroscopy (FTIR).

Suitable effective doses of the omega-3 lc-PUFA compositions for use in the methods described herein range from about 1 g per day to about 10 g per day; from about 2 g to about 9 g per day; from about 3 g to about 8 g per day; from about 4 g to about 7 g per day; from about 5 g to about 6 g per day, depending upon the composition, the dosage form, patient body size, and the seriousness of the condition to be treated.

In a particular series of embodiments, the effective dosage amounts of the omega-3 lc-PUFA compositions range from about 1 g to about 4 g per day. Accordingly, a suitable effective dose of the omega-3 lc-PUFA compositions described herein is at least about 1 g/day, at least about 2 g/day, at least about 3 g/day, or at least about 4 g/day.

5.2.5. Dosage Schedule

The effective amount of omega-3 lc-PUFA compositions described herein refer to total amounts administered per day. An effective dose can be administered in a single dose or as a divided dose. In one embodiment, an effective dose is administered once about every 24 h. In another embodiment, an effective dose is split, and administered in two doses over the course of 24 h. In a particular embodiment, an effective dose is administered once per day, at or near the same time every day. In another particular embodiment, the effective dose is administered in two doses over 24 h, each at or near the same time every day.

If the amount required to be administered is sufficiently small, the dose may be administered using a single unit dosage form, e.g., in one capsule or tablet. In typical embodiments, each administration requires multiple unit dosage forms, e.g., 2, 3 or 4 capsules.

In some embodiments, the omega-3 lc-PUFA composition is first administered in advance of the antiplatelet therapy, for a time sufficient to reduce plasma arachidonic acid levels sufficiently to treat, reverse, inhibit, or prevent resistance to antiplatelet therapy. In certain embodiments, the omega-3 lc-PUFA composition is administered for at least 1 day, for at least 2 days, for at least 3 days, for at least 4 days, for at least 5 days, for at least 6 days, for at least 1 week, for at least 2 weeks, for at least 3 weeks, or for at least 1 month or more in advance of the antiplatelet therapy. In other embodiments, the omega-3 lc-PUFA composition is first administered concurrently with initiation of the antiplatelet therapy.

In various embodiments, the omega-3 lc-PUFA composition is administered for the duration of the antiplatelet therapy. In some embodiments, the omega-3 lc-PUFA composition is first administered in advance of antiplatelet therapy and is then administered concurrently with the antiplatelet therapy.

In certain embodiments, the omega-3 lc-PUFA composition is administered for 2 weeks, 4 weeks, 6 weeks, 8 weeks, 12 weeks, 4 months, 6 months, 8 months, 12 months, 24 months or longer, depending on the nature and severity of the condition.

In certain embodiments, the omega-3 compositions are administered with food, typically with breakfast and/or dinner. In other embodiments, the compositions are administered to a subject who is fasting.

In certain embodiments, the fatty acid compositions are optionally co-administered with one or more additional therapeutic agents other than antiplatelet agents, or provided in a unit dose pharmaceutical formulation with one or more additional therapeutic agents other than antiplatelet agents, where the one or more additional therapeutic agents is useful in reducing the occurrence of or preventing cardiovascular disease from occurring or progressing, or are effective in treating any of the underlying risk factors that are commonly associated with cardiovascular disease. Such additional therapeutic agents include, but are not limited to, cardiac glycosides (e.g., digoxin), antiarrhythmic agents (e.g., procainamide, verapamil, propanolol), antianginal agents (e.g., nitroglycerin, diltiazem), antihypertensive agents (e.g., hydrochlorothiazide, captopril, prazocin), anticoagulant agents (e.g., coumadin, heparin), thrombolytic agents (e.g., alteplase, streptokinase, urokinase), cholesterol lowering agents (e.g., statins, fibrates, nicotinic acid), and pharmaceutically acceptable esters, derivatives, conjugates, precursors or salts thereof, and combinations thereof.

An effective amount of an additional therapeutic agent will be known to the art depending on the agent. However, it is well within the skilled artisan's purview to determine the additional therapeutic agent's optimal effective-amount range.

5.3. Omega-3 lc-PUFA Compositions

5.3.1. General Compositional Aspects

In certain embodiments, the composition comprising omega-3 lc-PUFAs (“omega-3 composition”) for use in the methods described herein comprises fatty acids in the form of a pharmaceutically acceptable ester, such as a C₁-C₅ alkyl ester, e.g., methyl ester, ethyl ester, propyl ester, butyl ester and the like. In particular embodiments, the fatty acids in the composition are in the form of an ethyl ester. In other particular embodiments, the fatty acids in the composition are in the free acid form. In certain embodiments, the fatty acids in the composition are salts of free acids. Unless otherwise noted, reference to a specific species of omega-3 or omega-6 fatty acid, such as “EPA,” “DHA,” and the like, are meant to encompass free acid forms and salts thereof, pharmaceutically acceptable esters, amides, triglycerides, diglycerides, monoglycerides, phospholipids, and derivatives, including, but not limited to, alpha-substituted derivatives, conjugates, including, but not limited to, conjugates with active ingredients such as salicylates, fibrates, niacin, cyclooxygenase inhibitors, or antibiotics, or salts thereof, or mixtures of any of the foregoing.

The fatty acid content of the compositions described herein can be determined by any method known in the art. Exemplary methods for determining the fatty acid profile of a composition include, but are not limited to, chromatographic methods such as gas chromatography (GC), gas liquid chromatography (GLC), mass spectrometry (MS), high performance liquid chromatography (HPLC), reverse phase HPLC, thin layer chromatography (TLC), GC-MS and TLC-GLC, and the like, and spectroscopic methods such as nuclear magnetic resonance spectroscopy (NMR) and Fourier transform infrared spectroscopy (FTIR).

In certain embodiments, the composition comprises the omega-3 lc-PUFA species, EPA. In various embodiments, the composition comprises EPA and DHA. In a variety of embodiments, the composition comprises the omega-3 lc-PUFA species, docosapentaenoic acid (n-3) (“DPA”). In some embodiments, the composition comprises EPA, DHA, and DPA.

In various embodiments, the composition comprises EPA in an amount, as a percentage by area on GC chromatogram of all fatty acids in the composition (“% (a/a)”), of at least about 20%, at least about 25%, at least about 30%, at least about 35%, at least about 40%, at least about 45%, at least about 50%, at least about 55%, of at least about 60%, at least about 65%, at least about 70%, at least about 75%, at least about 80%, at least about 85%, at least about 90%, at least about 95%, at least about 96%, at least about 97% or at least about 98%, at least about 99% or even about 100%. In certain embodiments, the composition comprises EPA in an amount ranging between any of the foregoing values.

In certain embodiments, the composition comprises DHA in an amount, as a percentage by area on GC chromatogram of all fatty acids in the composition, of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or of at least about 95%. In certain embodiments, the composition comprises DHA in an amount ranging between any of the foregoing values.

In certain embodiments, the composition comprises DHA in an amount, as a percentage by area on GC chromatogram of all fatty acids in the composition, of not more than about 10%, not more than about 9%, not more than about 8%, not more than about 7%, not more than about 6%, not more than about 5%, not more than about 4%, not more than about 3%, not more than about 2%, not more than about 1%, or not more than about 0.5% of total fatty acids in the composition. In a particular embodiment, the composition comprises no detectable DHA

In certain embodiments, the composition comprises EPA in an amount, as a percentage by area on GC chromatogram of all fatty acids in the composition, of at least about 20%, at least about 25%, at least about 30%, at least about 35%, at least about 40%, at least about 45%, at least about 50%, at least about 55%, of at least about 60%, at least about 65%, at least about 70%, at least about 75%, at least about 80%, at least about 85%, at least about 90% or of at least about 95% by weight of total fatty acids in the composition, and further comprises DHA in an amount such that the total EPA+DHA in the composition, as a percentage by area on GC chromatogram of all fatty acids in the composition, is at least about 20%, at least about 30%, at least about 40%, at least about 50%, at least about 55%, of at least about 60%, at least about 65%, at least about 70%, at least about 75%, at least about 80%, at least about 85%, at least about 90%, at least about 95%, at least about 96%, at least about 97%, at least about 98%, at least about 99% or even about 100% by weight of total fatty acids in the composition.

In certain embodiments, the composition comprises EPA in the ethyl ester form (eicosapent ethyl) in an amount, as a percentage by area on GC chromatogram of all fatty acids in the composition, of at least about 96%, with no detectable DHA. In a particular embodiment, the composition comprises EPA in the ethyl ester form (eicosapent ethyl) in an amount, as a percentage by weight of all fatty acids in the composition, of at least about 96%, with no detectable DHA. In a particular embodiment, the composition is Vascepa (Amarin Corporation).

In certain embodiments, the composition comprises EPA and DHA in a ratio (either a ratio of percentage by area on GC chromatogram, or as a ratio by weight) of about 1:1, of about 1.25:1, of about 1.5:1, of about 1.75:1, of about 2:1, of about 2.25:1, of about 2.5:1, of about 2.75:1, of about 3:1, of about 3.25:1, of about 3.5:1, of about 3.75:1, of about 4:1, of about 4.25:1, of about 4.5:1, of about 4.75:1 or of about 5:1. In particular embodiments, the composition comprises EPA and DHA in a ratio of about 2:1, of about 3:1, of about 1.24:1, of about 4:1 or of about 4.1:1. In a particular embodiment, the composition comprises EPA and DHA in the ethyl ester form in a weight ratio of about 1.24:1 to about 1.43.

In certain embodiments, the composition comprises EPA and DHA in the free acid form in weight ratios ranging from about 2:1 to about 4:1.

In some embodiments, the composition comprises EPA in the ethyl ester form in an amount of from about 40% to about 50% by weight, and DHA in the ethyl ester form in an amount of from about 30% to about 45% by weight of total fatty acids in the composition. In other embodiments, the composition comprises EPA in the ethyl ester form in an amount of from about 43% to about 49.5% by weight, and DHA in the ethyl ester form in an amount of from about 34.7% to about 40.3% by weight of total fatty acids in the composition. In other embodiments, the composition comprises EPA ethyl ester in an amount of from about 70% to about 80% by weight, and DHA in an amount of from about 10% to about 20% by weight. In still other embodiments, the pharmaceutical composition comprises EPA in the ethyl ester form in an amount of at least about 96% by weight, and no detectable DHA. In particularly preferred embodiments, the composition comprises EPA in the free acid form in an amount of from about 50% to about 60% by weight, and DHA in the free acid form in an amount of from about 15% to about 25% by weight of total fatty acids in the composition.

In various embodiments, the composition comprises at least one species of omega-3 or omega-6 fatty acid other than EPA or DHA in an amount of not more than about 30%, not more than about 25%, not more than about 20%, not more than about 15%, not more than about 10%, not more than about 9%, not more than about 8%, not more than about 7%, not more than about 6%, not more than about 5%, not more than about 4%, not more than about 3%, not more than about 2%, or not more than about 1%, by weight of the total weight of fatty acids in the composition.

In certain embodiments, the composition comprises fatty acid species other than EPA and DHA in an amount of from about 12% to about 20% by weight of total weight of fatty acids in the composition. Illustrative examples of such species include saturated fatty acids, mono-unsaturated fatty acids, species of omega-6 PUFAs such as arachidonic acid (AA, C20:4), linoleic acid (LA, C18:2), γ-linolenic acid (GLA, C20:3), and α-linolenic acid (ALA, C18:3), and omega-3 fatty acids such as stearidonic acid (STA, C18:4), eicosatrienoic acid (ETA, C20:3), eicosatetraenoic acid (ETE, C20:4), docosapentaenoic acid (DPA, C22:5), heneicosapentaenoic acid (HPA, C21:5), tetracosapentaenoic acid (C24:5) and tetracosahexaenoic acid (C24:6).

In certain embodiments, the fatty acid species other than EPA and DHA is in the ester form. In particular embodiments, the composition comprises DPA, STA, HPA, ETE and ALA in the ethyl ester form in a combined total amount of from about 12% to about 20% by weight of total weight of fatty acids in the composition.

In certain embodiments, the composition comprises no detectable omega-3 fatty acids other than EPA and DHA. In various embodiments, the composition comprises omega-3 fatty acids other than DHA and EPA in an amount of not more than about 1% by weight, not more than about 2% by weight, not more than about 3% by weight, not more than about 4% by weight, not more than about 5% by weight, not more than about 6% by weight, not more than about 7% by weight, not more than about 8% by weight, not more than about 9% by weight, not more than about 10% by weight, not more than 11% by weight, not more than 12% by weight, not more than about 13% by weight, not more than about 14% by weight or not more than about 15% by weight, not more than about 16% by weight, not more than about 17% by weight, not more than about 18% by weight, not more than about 19% or not more than about 20% by weight of total fatty acids in the composition. In certain embodiments, the composition comprises omega-3 fatty acids other than EPA and DHA in an amount ranging between any of the foregoing values, e.g., 1%-15% by weight, 4%-12% by weight, 10%-15% by weight, 5%-10% by weight, 1%-4% by weight, and the like, of total fatty acids.

In certain embodiments, the composition comprises total omega-6 PUFAs in a combined amount of not more than about 20% by weight, not more than about 19% by weight, not more than about 18% by weight, not more than about 17% by weight, not more than about 16% by weight, not more than about 15% by weight, not more than about 14% by weight, not more than about 13% by weight, not more than about 12% by weight, not more than about 11% by weight, not more than about 10% by weight, not more than about 9% by weight, not more than about 8% by weight, not more than about 7% by weight, not more than about 6% by weight, not more than about 5% by weight, not more than about 4% by weight, not more than about 3% by weight, not more than about 2% by weight, not more than about 1% by weight, or not more than about 0.5% by weight of total fatty acids in the composition. In some embodiments, the composition comprises omega-6 fatty acids in a combined amount of not more than about 10% by weight of total fatty acids in the composition. In some embodiments, the composition comprises omega-6 fatty acids in a combined amount of not more than about 10% by chromatographic area, of total fatty acids in the composition.

In some embodiments, the composition comprises AA in an amount of not more than about 10% by weight, not more than about 9% by weight, not more than about 8% by weight, not more than about 7% by weight, not more than about 6% by weight, not more than about 5.5% by weight, not more than about 5% by weight, not more than about 4.5% by weight, not more than about 4% by weight, not more than about 3.5% by weight, not more than about 3% by weight, not more than about 2.5% by weight, not more than about 2% by weight, not more than about 1.5% by weight, not more than about 1% by weight or not more than about 0.5% by weight of total fatty acids in the composition. In certain embodiments, the composition comprises AA in an amount of not more than about 4.5% by weight of total fatty acids in the composition. In some embodiments, the composition comprises AA in an amount of not more than about 4.5% by chromatographic area of total fatty acids in the composition.

In various embodiments, the composition comprises other fatty acids, such as saturated fatty acids in an amount of not more than about 5% by weight, not more than about 4% by weight, not more than about 3% by weight, not more than about 2% by weight or not more than about 1% by weight of total fatty acids in the composition, and/or monounsaturated fatty acids in an amount of not more than about 7% by weight, not more than about 6% by weight, not more than about 5% by weight, not more than about 4% by weight, not more than about 3% by weight, not more than about 2% by weight or not more than about 1% by weight of total fatty acids in the composition. In some embodiments, the composition comprises unsaturated fatty acids other than polyunsaturated fatty acids and monounsaturated fatty acids in an amount of not more than about 7% by weight, not more than about 6% by weight, not more than about 5% by weight, not more than about 4% by weight, not more than about 3% by weight, not more than about 2% by weight, or not more than about 1% by weight of total fatty acids in the composition. In a particular embodiment, the composition comprises saturated fatty acids in an amount of not more than about 3% by weight, monounsaturated fatty acids in an amount of not more than 5% by weight, and unsaturated fatty acids other than omega-3 and omega-6 polyunsaturated fatty acids and monounsaturated fatty acids in an amount of not more than 5% by weight of total fatty acids in the composition. In another particular embodiment, the composition comprises saturated fatty acids in an amount of not more than about 3%, monounsaturated fatty acids in an amount of not more than 5%, and unsaturated fatty acids other than omega-3 and omega-6 polyunsaturated fatty acids and monounsaturated fatty acids in an amount of not more than 5% by chromatographic area of total fatty acids in the composition.

In some embodiments, the composition is Lovaza (GSK). In certain embodiments, the composition is Vascepa (Amarin Corporation). In certain embodiments, the composition is Omax3 (Cenestra Health).

The sources of the fatty acids for use in the pharmaceutical compositions described herein include, but are not limited to, fish oil, marine microalgae oils, plant oils or combinations thereof. In some embodiments, the fatty acids are derived from algae. In a particular embodiment, the source of the fatty acids for use in the pharmaceutical compositions described herein is fish oil. Because the fatty acids are derived from natural sources, in certain embodiments, the compositions include trace amounts of other substances derived from the source oil, such as fat soluble vitamins, e.g., vitamin A and/or vitamin D, and/or cholesterol.

The fatty acids for use in the compositions described herein can be isolated and purified by any method known in the art. In certain embodiments, where a composition of fatty acid esters is desired, the fatty acids are extracted and purified from marine oils by (i) refining and deodorizing crude marine oil triglycerides; (ii) esterifying the fatty acids; (iii) fractionating and concentrating the esters, e.g., by fractional distillation; (iv) removing saturated fatty acids and other contaminants; and (v) concentrating the fatty acid esters, e.g., by distillation, to achieve the final product. In certain embodiments, where a composition of free fatty acids is desired, the fatty acid esters obtained after step (iv) can be hydrolyzed, for example, by base hydrolysis, and then be further purified by fractional distillation. In other embodiments, the marine oil can be deacidified before the refining step by, for example, distillation or washing with sodium hydroxide, to remove the free fatty acids. Exemplary methods of obtaining fatty acid compositions are found, for example, in U.S. Pat. Nos. 5,656,667 and 6,630,188 to Norsk Hydro AS, U.S. Pat. No. 7,807,848 to Ocean Nutrition Canada, Ltd. And U.S. Pat. No. 7,119,118 to Laxdale Ltd.

5.3.2. n-3 FFA Compositions

As further described in Example 3, below, the inventors have further discovered that compositions comprising lc-omega-3 PUFAs in free acid form (“n-3 FFA compositions”) provide unprecedented potency in reducing AA plasma levels. Thus, in a particular series of embodiments, the fatty acid composition for use in the methods described herein comprises lc-PUFAs in the free acid foam.

In various embodiments, the n-3 FFA composition comprises EPA in an amount of at least about 50% (a/a). In certain embodiments, the n-3 FFA composition comprises DHA in an amount of at least about 15% (a/a).

In a variety of embodiments, the n-3 FFA composition comprises EPA and DHA, each in the range of ±3 standard deviations from its respective average, expressed as a percentage (a/a) of all fatty acids in the composition, as set forth in Table 1, below. In some embodiments, the n-3 FFA composition comprises EPA and DHA, each in a range of ±2 standard deviations from its respective average, as set forth in Table 1, below. In some embodiments, the n-3 FFA composition comprises EPA and DHA, each in the range of ±1 standard deviation from its respective average, as set forth in Table 1, below. In various embodiments, the n-3 FFA composition comprises EPA and DHA, each in an amount about equal to the respective average set forth in Table 1, below.

In various embodiments, the n-3 FFA composition further comprises DPA. In some embodiments, DPA is present in an amount of at least about 2.5% (a/a). In various embodiments, DPA is present in a range of ±3 standard deviations from its respective average, expressed as a percentage (a/a) of all fatty acids in the composition, as set forth in Table 1, below. In various embodiments, DPA is present in a range of ±2 standard deviations from its respective average, expressed as a percentage (a/a) of all fatty acids in the composition, as set forth in Table 1, below. In some embodiments, in a range of ±1 standard deviations from its respective average, expressed as a percentage (a/a) of all fatty acids in the composition, as set forth in Table 1, below. In various embodiments the n-3 FFA composition comprises DPA in an amount about equal to the average set forth in Table 1, below.

TABLE 1

| | | | | | | | | | | | 1S | 2S | 3S |
|---|---|---|---|---|---|---|---|---|---|---|---|---|---|
| Identity | Classification | STDEV | Average | −3SD | −2SD | −1SD | Average | +1SD | +2SD | +3SD | Delta | Delta | Delta |
| C18:2(n-6) | Linoleic acid | 0.088 | 0.605 | 0.342 | 0.430 | 0.517 | 0.605 | 0.693 | 0.780 | 0.868 | 0.175 | 0.351 | 0.526 |
| C18:3(n-6) | Gamma-linolenic acid | 0.028 | 0.154 | 0.069 | 0.098 | 0.126 | 0.154 | 0.182 | 0.210 | 0.239 | 0.056 | 0.113 | 0.169 |
| C18:3(n-3) | α-Linolenic acid | 0.064 | 0.428 | 0.234 | 0.299 | 0.363 | 0.428 | 0.492 | 0.557 | 0.621 | 0.129 | 0.258 | 0.387 |
| C18:4(n-3) | Moroctic acid | 0.250 | 1.563 | 0.811 | 1.062 | 1.312 | 1.563 | 1.813 | 2.063 | 2.314 | 0.501 | 1.002 | 1.502 |
| C20:2(n-6) | Eicosadienoic acid | 0.053 | 0.126 | −0.031 | 0.022 | 0.074 | 0.127 | 0.180 | 0.232 | 0.285 | 0.105 | 0.211 | 0.316 |
| C20:3(n-6) | Dihomo-gamma- | 0.055 | 0.444 | 0.278 | 0.334 | 0.389 | 0.444 | 0.500 | 0.555 | 0.610 | 0.111 | 0.221 | 0.332 |
| | linolenic acid | | | | | | | | | | | | |
| C20:4(n-6) | Arachidonic acid (AA) | 0.576 | 3.141 | 1.413 | 1.989 | 2.565 | 3.141 | 3.716 | 4.292 | 4.868 | 1.152 | 2.303 | 3.455 |
| C20:3(n-3) | Eicosatrienoic acid | 0.040 | 0.197 | 0.078 | 0.118 | 0.157 | 0.197 | 0.237 | 0.276 | 0.316 | 0.079 | 0.158 | 0.238 |
| C20:4(n-3) | Eicosatetraenoic acid | 0.243 | 2.191 | 1.464 | 1.706 | 1.949 | 2.191 | 2.434 | 2.676 | 2.919 | 0.485 | 0.970 | 1.455 |
| C20:5(n-3) | Eicosapentaenoic acid | 0.557 | 56.744 | 55.072 | 55.629 | 56.186 | 56.744 | 57.301 | 57.859 | 58.416 | 1.115 | 2.230 | 3.344 |
| | (EPA) | | | | | | | | | | | | |
| C21:5(n-3) | Heneicosapentaenoic | 0.252 | 2.610 | 1.853 | 2.105 | 2.358 | 2.610 | 2.863 | 3.115 | 3.368 | 0.505 | 1.010 | 1.515 |
| | acid | | | | | | | | | | | | |
| C22:5(n-6) | Docosapentaenoic | 0.206 | 0.569 | −0.050 | 0.156 | 0.362 | 0.569 | 0.775 | 0.981 | 1.188 | 0.413 | 0.825 | 1.238 |
| | acid (n-6) | | | | | | | | | | | | |
| C22:5(n-3) | Docosapentaenoic | 1.058 | 5.307 | 2.132 | 3.190 | 4.249 | 5.307 | 6.365 | 7.424 | 8.482 | 2.117 | 4.234 | 6.351 |
| | acid (n-3) (DPA) | | | | | | | | | | | | |
| C22:6(n-3) | Docosahexaenoic acid | 0.749 | 19.930 | 17.684 | 18.433 | 19.181 | 19.930 | 20.678 | 21.427 | 22.175 | 1.497 | 2.994 | 4.491 |
| | (DHA) | | | | | | | | | | | | |

In various embodiments, the n-3 FFA composition further comprises AA. In certain embodiments, AA is present in an amount ranging from ±3 standard deviations of the average, as set forth in Table 1 above. In some embodiments, AA is present in an amount ranging from ±2 standard deviations of the average, as set forth in Table 1. In certain embodiments, AA is present in an amount ranging from ±1 standard deviation of the average in Table 1. In some embodiments, AA is present in an amount of about the average set forth in Table 1. In certain embodiments, AA is present in an amount of no more than about 5% (a/a) or 5% (w/w). In various embodiments, AA is present in an amount of no more than about 4.5% (a/a) or 4.5% (w/w).

In certain embodiments, the n-3 FFA composition comprises EPA, DHA, DPA, AA, and one or more additional lc-PUFA species recited in Table 1. In specific embodiments, the n-3 FFA composition comprises all of the lc-PUFA species recited in Table 1. In a particular embodiment, the n-3 FFA composition has the composition set forth in Table 2, below.

In various embodiments, the composition comprises EPA+DHA in a total amount, calculated as a percentage by mass of all fatty acids in the composition, of about 70.0-80.0% (m/m). In certain embodiments, the composition comprises about 75.0% (m/m) EPA plus DHA. In typical embodiments, total omega-3 fatty acids comprise from about 80.0-about 95% (m/m) of all fatty acids in the pharmaceutical composition.

The composition comprises, in typical embodiments, no more than about 3.0% (a/a) saturated fatty acids, no more than about 5.0% (a/a) mono-unsaturated fatty acids, and no more than about 0.1 ppm ethyl carbamate. In various embodiments, the composition comprises less than 0.1 ppm ethyl carbamate.

Processes for producing the n-3 FFA compositions described herein and presented in Tables 1 and 2 are described in U.S. provisional application No. 61/583,796, filed Jan. 6, 2012, the disclosure of which is incorporated herein by reference in its entirety.

5.3.3. Drug Product and Dosage Forms

In certain embodiments, the pharmaceutical compositions comprising omega-3 lc-PUFAs used in the methods described herein, including n-3 FFA compositions, contain one or more pharmaceutically acceptable carriers, excipients or stabilizers (referred to as “excipients” herein) typically employed in the art, i.e., fillers, stabilizers, extenders, binders, humidifiers, surfactants, lubricants, preservatives, antioxidants, flavorants, colorants and other miscellaneous additives. Specific examples of pharmaceutically acceptable carriers and excipients that can be used to formulate oral dosage forms are described in the Handbook of Pharmaceutical Excipients, American Pharmaceutical Association (1986) and in Remington's Pharmaceutical Sciences, 16th edition (Osol, ed. 1980). Such additives must be nontoxic to the recipients at the dosages and concentrations employed. An excipient can be inert or it can possess pharmaceutical benefits.

In certain embodiments, the omega-3 compositions described herein comprise an antioxidant. Suitable antioxidants include, but are not limited to, tocopherols, such as α-tocopherol, β-tocopherol, γ-tocopherol, δ-tocopherol, and tocotrienols, such as α-tocotrienol, β-tocotrienol, γ-tocotrienol and δ-tocotrienol. In certain embodiments, an antioxidant can be present in the composition in an amount of from about 0.1% to about 0.5% by weight, of from about 0.15% to about 0.25% by weight, of from about 0.2% to about 0.4% by weight, or of from about 0.25% to about 0.35% of total fatty acids in the composition. In one embodiment, the antioxidant is α-tocopherol that is present in an amount of from about 0.4% to about 0.44% by weight of the composition. In another embodiment, the α-tocopherol is present in the composition in an amount of about 0.27% to about 0.33% by weight of the composition.

Excipients are selected with respect to the intended form of administration and consistent with conventional pharmaceutical practices. Preferably, the compositions are administered orally, e.g., in tablets, capsules, powders, syrups, suspensions, and the like.

In particular embodiments, the pharmaceutical dosage form is a capsule. In certain embodiments, the dosage form is a gelatin capsule. In certain embodiments, the gelatin capsule is a hard gelatin capsule. In other embodiments, the dosage form is a soft gelatin capsule. A gelatin capsule for encapsulating the pharmaceutical compositions described herein can be made from Type A gelatin (i.e., gelatin extracted by a process comprising an acid pre-treatment of a collagen source) made from, e.g., pig skin (porcine type A gelatin), or from Type B gelatin (gelatin extracted by a process comprising an alkaline pre-treatment of a collagen source), such as bovine type B gelatin. Sources of collagen for the production of gelatin include, but are not limited to, pigs, cows, and fish. Capsules can also be made from substances that are not animal by-products such as agar-agar, carrageenan, pectin, konjak, guar gum, food starch, modified corn starch, potato starch, and tapioca. Non-animal sources of materials that can be used to make capsules are described in U.S. Patent Publication No. 2011/0117180, assigned to Ocean Nutrition Canada Ltd.

In particular embodiments, the dosage form of the pharmaceutical compositions described herein is a soft gelatin capsule made from Type A porcine gelatin. In specific embodiments, the dosage form is a soft gelatin capsule made from Type A porcine gelatin, with the active fill being an n-3 FFA composition as described in Section 5.3.2 above.

In addition to gelatin or a non-animal gelling agent, in particular embodiments a soft gelatin capsule shell is used that contains a plasticizer and water. Plasticizers for use in soft gelatin capsules include, but are not limited to, small polyhydroxy compounds such as glycerol, sorbitol, propylene glycol, sucrose, maltitol and mixtures thereof. In certain embodiments, the gelatin capsule contains one or more substances selected from a preservative such as methyl paraben or propylmethyl paraben, a colorant, an opacifying agent such as titanium dioxide, a flavoring agent, a sugar, a chelating agent and a medicament. In certain embodiments, the gelatin capsule comprises water in an amount of at least about 1% by weight, of at least about 2% by weight, of at least about 3% by weight, of at least about 4% by weight, of at least about 5% by weight, of at least about 6% by weight, of at least about 7% by weight, of at least about 8% by weight, of at least about 9% by weight or of at least about 10% by weight of the composition. In certain embodiments, the gelatin capsule comprises water in an amount ranging between any of the foregoing values, e.g., 1%-5% by weight, 2%-8% by weight, 6%-10% by weight, 5%-10% by weight, and the like. In a particular embodiment, the gelatin capsule comprises water in an amount of between about 6% and about 10% by weight of the composition. In some embodiments, the gelatin capsule comprises a plasticizer in an amount of not more than about 0.1%, of not more than about 0.2%, of not more than about 0.3%, of not more than about 0.4%, of not more than about 0.5%, of not more than about 0.6%, of not more than about 0.7%, of not more than about 0.8%, of not more than about 0.9% or of not more than about 1% by weight of the composition.

In certain embodiments, the gelatin capsule is uncoated. In other embodiments, the gelatin capsule is coated.

In various coated embodiments, the capsule has an enteric coating, to delay release of the fatty acid composition until after passage through the stomach. In other embodiments, the capsule is coated so as to delay release of the fatty acid composition for at least 30 minutes after ingestion. In various embodiments, release of the fatty acid composition is delayed for about 30 minutes to about 60 minutes after ingestion. Suitable coatings for achieving delayed release of the fatty acid composition are known to one of skill in the art and include coatings that are resistant to dissolution in a time, but not pH, dependent manner. In a particular embodiment, the gelatin capsule is coated with a poly(ethylacrylate-methylacrylate) polymer. In one embodiment, the dosage form is a soft gelatin capsule coated with a neutral polyacrylate such as poly(ethylacrylate-methylmethacrylate), such as Eudragit NE 30-D (Rohm Pharma GmbH), which has an average molecular weight of about 800,000. In a particular embodiment, the dosage form is a coated Type A porcine soft gelatin capsule as described in U.S. Pat. No. 7,960,370 to Tillotts Pharma AG.

In some embodiments, the dosage form comprises 250 mg of a composition comprising omega-3 PUFAs. In various embodiments, the dosage form is selected from a 250-mg dosage form, a 300-mg dosage form, a 350-mg dosage form, a 400-mg dosage form, a 450-mg dosage form, a 500-mg dosage form, a 600-mg dosage form, a 700-mg dosage form, an 800-mg dosage form, a 900-mg dosage form, a 1-g dosage form, a 1.2-g dosage form and a 1.5-g dosage form. In some embodiments, the dosage form is a 1.5-g dosage form. In particular embodiments, the dosage form is a 1-g dosage form. In certain embodiments, the 1-g dosage form is a coated soft porcine Type A gelatin capsule as described above. In certain embodiments, the 1-g dosage form comprises total omega-3 fatty acids in an amount of at least about 800 mg, of at least about 825 mg, of at least about 850 mg, of at least about 875 mg, of at least about 900 mg, of at least about 925 mg, of at least about 950 mg, of at least about 960 mg, or of at least about 975 mg per 1-g dosage form.

In certain embodiments, the 1-g dosage form comprises total omega-3 fatty acids in an amount ranging between any of the foregoing values, e.g., 800 mg-950 mg, 875 mg-900 mg, 900 mg-975 mg, and the like. In one particular embodiment, the dosage form is a 1-g soft gelatin capsule that comprises at least about 900 mg of the ethyl esters of total omega-3 fatty acids. In another particular embodiment, the dosage form is a 1-g soft gelatin capsule that comprises from about 800 mg to about 950 mg of total omega-3 fatty acids in the free acid form. In yet another embodiment, the dosage form is a 500-mg capsule that comprises from about 400 mg to about 495 mg, from about 425 mg to about 480 mg, or from about 450 mg to about 490 mg of the ethyl ester form of EPA. In still other embodiments the dosage form is a 1.5-g capsule that comprises at least about 1,300 mg, at least about 1,350 mg, at least about 1,400 mg, or at least about 1,450 mg of EPA and DHA ethyl esters.

In a particular series of embodiments, the dosage form is a porcine Type A soft gelatin capsule filled with an n-3 FFA composition as described in Section 5.3.2, and coated with Eudragit NE-30D formulated so as to delay capsule rupture for at least 30 minutes when tested in vitro in USP apparatus 2 at 37° C. at pH 5.5.

5.3.4. Dosage Forms Comprising an Omega-3 lc-PUFA Composition and at Least One Antiplatelet Agent

In another aspect, dosage forms that comprise (a) a composition comprising omega-3 lc-PUFAs and (b) one or more compositions for antiplatelet therapy (“anti-platelet agent”) are provided.

Antiplatelet agents suitable for inclusion in such dual compositions include adenosine diphosphate (ADP) receptor inhibitors, such as clopidogrel (Plavix®), ticlopidine (Ticlid®), prasugrel (Effient®), and ticagrelor (Brilinta®); phosphodiesterase inhibitors such as cilostazol (Pletal®); glycoprotein IIb/IIIa inhibitors such as abciximab (ReoPro®), eptifibatide (Integrilin®) and tirofiban (Aggrastat®). In various embodiments, the antiplatelet therapy agent is an adenosine reuptake inhibitor such as dipyridamole (Persantine®); a thromboxane inhibitor, e.g., thromboxane synthase inhibitors or thromboxane receptor antagonists such as terutroban, and combinations thereof.

In certain embodiments, the antiplatelet therapy agent is a non-steroidal anti-inflammatory drug selected from the group consisting of aspirin, aloxiprin, benorylate, diflunisal, ethenzamide, magnesium salicylate, methyl salicylate, salsalate, salicin, salicylamide, sodium salicylate, arylalkanoic acids, diclofenac, aceclofenac, acemetacin, alclofenac, bromfenac, etodolac, indometacin, indometacin farnesil, mabumetone, oxametacin, proglumetacin, sulindac, tolmetin, ibuprofen, alminoprofen, benoxaprofen, carprofen, dexibuprofen, dexketoprofen, fenbufen, fenoprofen, flunoxaprofen, flurbiprofen. ibuproxam, indoprofen, ketoprofen, ketorolac, loxoprofen, miroprofen, naproxen, oxaprozin, pirprofen, suprofen, tarenflurbil, tiaprofenic acid, mefenamic acid, flufenamic acid, meclofenamic acid, tolfenamic acid, phenylbutazone, ampyrone, azapropazone, clofezone, kebuzone, metamizole, mofebutazone, oxyphenbutazone, phenazone, sulfinpyrazone, piroxicam, droxicam, lornoxicam, meloxicam, tenoxicamm ampiroxicam, celecoxib, deracoxib, etoricoxib, firocoxib, lumiracoxib, parecoxib, rofecoxib, valdecoxib, nimesulide, naproxcinod, fluproquazone, and combinations thereof.

In particular embodiments, the antiplatelet agent is clopidogrel. In some embodiments, the antiplatelet agent is aspirin. In certain embodiments, the antiplatelet agent is a combination of clopidogrel and aspirin.

In some embodiments, the omega-3 lc-PUFAs and antiplatelet therapy are present in a range of about 1:1000 to about 1000:1 by weight, preferably about 200:1 to about 200:1 by weight. In some embodiments, the omega-3 lc-PUFAs may be present in an amount from about 1 mg to about 3000 mg, more preferably from about 500 mg to about 2000 mg. In some embodiments, the antiplatelet therapy may be present in an amount from about 1 mg to about 1000 mg, more preferably from about 5 mg to about 500 mg, and even more preferably from about 5 mg to about 100 mg. In some preferred embodiments, the antiplatelet therapy is clopidogrel.

In certain embodiments, the omega-3 lc-PUFA composition is encapsulated as above-described, and the one or more antiplatelet agents is coated on the capsule. Techniques for coating active pharmaceutical compositions on capsules are described, e.g., U.S. Patent Application Pub. No. 2007/0212411, incorporated herein by reference in its entirety.

The one or more coatings on the capsule may be applied by any conventional technique including, but not limited to, pan coating, fluid bed coating or spray coating. The coating(s) may be applied, for example, as a solution, suspension, spray, dust or powder. In some embodiments the average thickness of the coating layer is from 5-400 microns, preferably 10-200 microns, more preferably 20-100 microns, most preferably 40-80 microns.

In certain embodiments, the coating layer comprises a polymer. Suitable polymers include any pharmaceutically acceptable polymers known to those of skill in the art. Preferred polymers include, but are not limited to, cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions and combinations thereof, preferably hydroxpropyl cellulose, ethyl cellulose, and mixtures thereof.

### 6. EXAMPLES

### 6.1. Example 1

### Pharmaceutical Compositions of PUFAs In Free Acid Form (“n-3 FFA Compositions”)

Ten exemplary production batches of pharmaceutical compositions comprising PUFAs in free acid form (“n-3 FFA compositions”) were prepared. Average, standard deviation (STDEV, or SD), and Delta (the absolute difference between +1 SD and −1 SD (“1SDelta”); between +2 and −2 SD (“2SDelta”); and between +3 and −3 SD (“3SDelta”)) were calculated for various lc-PUFA species of the omega-3 (“n-3”) and omega-6 (“n-6”) series. Results are shown in Table 1, reproduced below.

TABLE 1

| | | | | | | | | | | | 1S | 2S | 3S |
|---|---|---|---|---|---|---|---|---|---|---|---|---|---|
| Identity | Classification | STDEV | Average | −3SD | −2SD | −1SD | Average | +1SD | +2SD | +3SD | Delta | Delta | Delta |
| C18:2(n-6) | Linoleic acid | 0.088 | 0.605 | 0.342 | 0.430 | 0.517 | 0.605 | 0.693 | 0.780 | 0.868 | 0.175 | 0.351 | 0.526 |
| C18:3(n-6) | Gamma-linolenic acid | 0.028 | 0.154 | 0.069 | 0.098 | 0.126 | 0.154 | 0.182 | 0.210 | 0.239 | 0.056 | 0.113 | 0.169 |
| C18:3(n-3) | α-Linolenic acid | 0.064 | 0.428 | 0.234 | 0.299 | 0.363 | 0.428 | 0.492 | 0.557 | 0.621 | 0.129 | 0.258 | 0.387 |
| C18:4(n-3) | Moroctic acid | 0.250 | 1.563 | 0.811 | 1.062 | 1.312 | 1.563 | 1.813 | 2.063 | 2.314 | 0.501 | 1.002 | 1.502 |
| C20:2(n-6) | Eicosadienoic acid | 0.053 | 0.126 | −0.031 | 0.022 | 0.074 | 0.127 | 0.180 | 0.232 | 0.285 | 0.105 | 0.211 | 0.316 |
| C20:3(n-6) | Dihomo-gamma- | 0.055 | 0.444 | 0.278 | 0.334 | 0.389 | 0.444 | 0.500 | 0.555 | 0.610 | 0.111 | 0.221 | 0.332 |
| | linolenic acid | | | | | | | | | | | | |
| C20:4(n-6) | Arachidonic acid (AA) | 0.576 | 3.141 | 1.413 | 1.989 | 2.565 | 3.141 | 3.716 | 4.292 | 4.868 | 1.152 | 2.303 | 3.455 |
| C20:3(n-3) | Eicosatrienoic acid | 0.040 | 0.197 | 0.078 | 0.118 | 0.157 | 0.197 | 0.237 | 0.276 | 0.316 | 0.079 | 0.158 | 0.238 |
| C20:4(n-3) | Eicosatetraenoic acid | 0.243 | 2.191 | 1.464 | 1.706 | 1.949 | 2.191 | 2.434 | 2.676 | 2.919 | 0.485 | 0.970 | 1.455 |
| C20:5(n-3) | Eicosapentaenoic acid | 0.557 | 56.744 | 55.072 | 55.629 | 56.186 | 56.744 | 57.301 | 57.859 | 58.416 | 1.115 | 2.230 | 3.344 |
| | (EPA) | | | | | | | | | | | | |
| C21:5(n-3) | Heneicosapentaenoic | 0.252 | 2.610 | 1.853 | 2.105 | 2.358 | 2.610 | 2.863 | 3.115 | 3.368 | 0.505 | 1.010 | 1.515 |
| | acid | | | | | | | | | | | | |
| C22:5(n-6) | Docosapentaenoic | 0.206 | 0.569 | −0.050 | 0.156 | 0.362 | 0.569 | 0.775 | 0.981 | 1.188 | 0.413 | 0.825 | 1.238 |
| | acid (n-6) | | | | | | | | | | | | |
| C22:5(n-3) | Docosapentaenoic | 1.058 | 5.307 | 2.132 | 3.190 | 4.249 | 5.307 | 6.365 | 7.424 | 8.482 | 2.117 | 4.234 | 6.351 |
| | acid (n-3) (DPA) | | | | | | | | | | | | |
| C22:6(n-3) | Docosahexaenoic acid | 0.749 | 19.930 | 17.684 | 18.433 | 19.181 | 19.930 | 20.678 | 21.427 | 22.175 | 1.497 | 2.994 | 4.491 |
| | (DHA) | | | | | | | | | | | | |

### 6.2. Example 2

### Elevated Baseline AA Levels Correlate with Genotype at Certain SNPs in the FADS1 and FADS2 Genes, and can be Reduced with Clinically Relevant Doses of n-3 FFA Compositions

STUDY DRUG (Epanova®)—Type A porcine soft gelatin capsules were prepared, each containing one gram (1 g) of an omega-3 FFA composition (“API”). The capsules were coated with Eudragit NE 30-D (Evonik Industries AG). The API had the composition given in Table 2, below (Omefas Lot #36395).

TABLE 2

| Identity | Common name | area % |
|---|---|---|
| C18:2(n-6) | Linoleic acid | 0.493 |
| C18:3(n-6) | Gamma-linolenic acid | 0.139 |
| C18:3(n-3) | α-Linolenic acid | 0.337 |
| C18:4(n-3) | Moroctic acid | 1.673 |
| C20:2(n-6) | Eicosadienoic acid | 0.132 |
| C20:3(n-6) | Dihomo-gamma-linolenic acid | 0.389 |
| C20:4(n-6) | Arachidonic acid (AA) | 2.446 |
| C20:3(n-3) | Eicosatrienoic acid | 0.254 |
| C20:4(n-3) | Eicosatetraenoic acid | 2.019 |
| C20:5(n-3) | Eicosapentaenoic acid (EPA) | 57.635 |
| C21:5(n-3) | Heneicosapentaenoic acid | 2.752 |
| C22:5(n-6) | Docosapentaenoic acid (n-6) | 0.172 |
| C22:5(n-3) | Docosapentaenoic acid (n-3) (DPA) | 6.224 |
| C22:6(n-3) | Docosahexaenoic acid (DHA) | 19.647 |

STUDY DESIGN—This study (OM-007) employed an open-label, 2-way crossover design intended primarily to assess PK interaction between simvastatin and Epanova®. Each Epanova® dose consisted of 1 g of n-3 FFA composition (see Table 2) encapsulated in a porcine type A soft gelatin capsule coated with Eudragit NE-30D (Epanova®).

Treatment condition “A” consisted of co-administration of an oral dose of 40 mg of simvastatin (1 tablet), 81 mg of aspirin (1 tablet) and 4 g (4 capsules) of Epanova®, once a day (every 24 hours) with 240 mL of water on the mornings of Days 1 to 14, for a total of 14 doses, under fasting conditions. Treatment condition “B” consisted of administration of an oral dose of 40 mg of simvastatin (1 tablet) and 81 mg of aspirin (1 tablet) once a day (every 24 hours) with 240 mL of water on the mornings of Days 1 to 14, for a total of 14 doses, under fasting conditions. There was a 14 Day washout between treatments.

A total of 52 subjects were enrolled and randomized with respect to order of treatment. Blood was drawn for plasma fatty acid levels (EPA, DHA, AA) at check-in (day −1) and at check-out (day 15) following the treatment arm with Epanova® (treatment “A”). Genotyping was performed at various previously identified SNPs, including SNPs in the FADS1 gene, including a SNP associated with conversion of DGLA to AA (SNP rs174537), the FADS2 gene, and Scd-1 gene.

FIGS. 2-24 depict arachidonic acid (AA) plasma levels grouped according to genotype at the respectively identified SNPs, at (A) baseline (in μg/mL), and (B) day 15 of Treatment “A” (in percent change from baseline). For each genotype, the interquartile range is indicated by a box, the median is indicated by a horizontal line in the interior of the interquartile box, and the mean is represented by a diamond. Outliers are represented by open circles. The whiskers extend to the minimum and maximum non-outlier value. Score 1 identifies subjects who are homozygous at the major (most prevalent) allele; Score 3 identifies subjects homozygous at the minor allele; and Score 2 represents heterozygotes. Table 3, below, identifies the gene with which each of the tested SNPs is associated, and the respective Figure in which results are plotted.

TABLE 3

| Figure | SNP | Gene |
|---|---|---|
| 2 | rs174537 | FADS1 |
| 3 | rs174554 | FADS1 |
| 4 | rs174546 | FADS1 |
| 5 | rs102275 | FADS1 |
| 6 | rs174568 | FADS2 |
| 7 | rs1535 | FADS2 |
| 8 | rs174583 | FADS2 |
| 9 | rs17156535 | FADS3 |
| 10 | rs13966 | RAB3IL1 |
| 11 | rs174468 | FADS2 |
| 12 | rs174575 | FADS2 |
| 13 | rs174579 | FADS2 |
| 14 | rs174589 | FADS2 |
| 15 | rs174602 | FADS2 |
| 16 | rs174627 | FADS2 |
| 17 | rs2072114 | FADS2 |
| 18 | rs412334 | FEN1 |
| 19 | rs422249 | FADS2 |
| 20 | rs472031 | FADS2 |
| 21 | rs526126 | FADS2 |
| 22 | rs528285 | FADS2 |
| 23 | rs74557970 | FADS3 |
| 24 | rs7482316 | FADS2 |

As shown in FIG. 2A, subjects homozygous for the minor allele at FADS1 SNP rs174537 (genotype: GG) have higher median and mean baseline AA levels, and therefore have greater potential resistance to anti-platelet therapies. As shown in FIG. 2B, these individuals have a greater percentage reduction in AA levels after 14 day treatment with 4 g/day Epanova® compared to the other genotypes. Similar results are observed for FADS1 SNPs rs174554 (FIGS. 3A and 3B), rs174546 (FIGS. 4A and 4B), and rs102275 (FIGS. 5A and 5B).

As shown in FIG. 6A, subjects homozygous for the minor allele at FADS2 SNP rs174568 (genotype: CC) have higher median and mean baseline AA levels, and therefore have greater potential resistance to anti-platelet therapies. As shown in FIG. 6B, these individuals have a greater percentage reduction in AA levels after 14 day treatment with 4 g/day Epanova® compared to the other genotypes. Similar results are observed for FADS2 SNP rs1535 (FIGS. 7A and 7B) and rs174583 (FADS2 intronic).

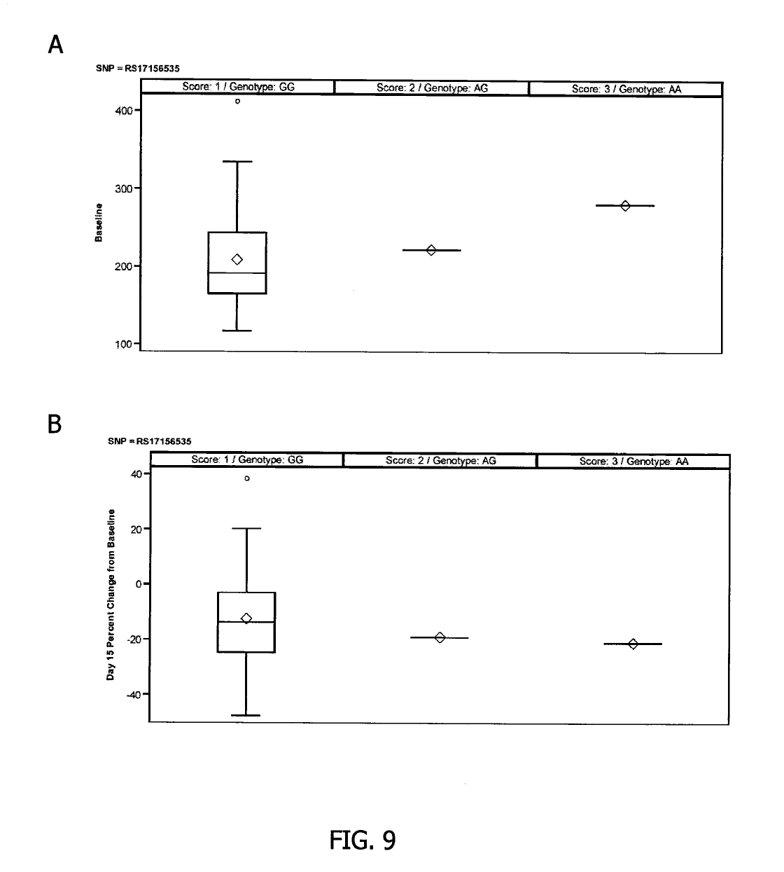

FIG. 9 shows analogous results for rs17156535, a SNP associated with the FADS3 gene.

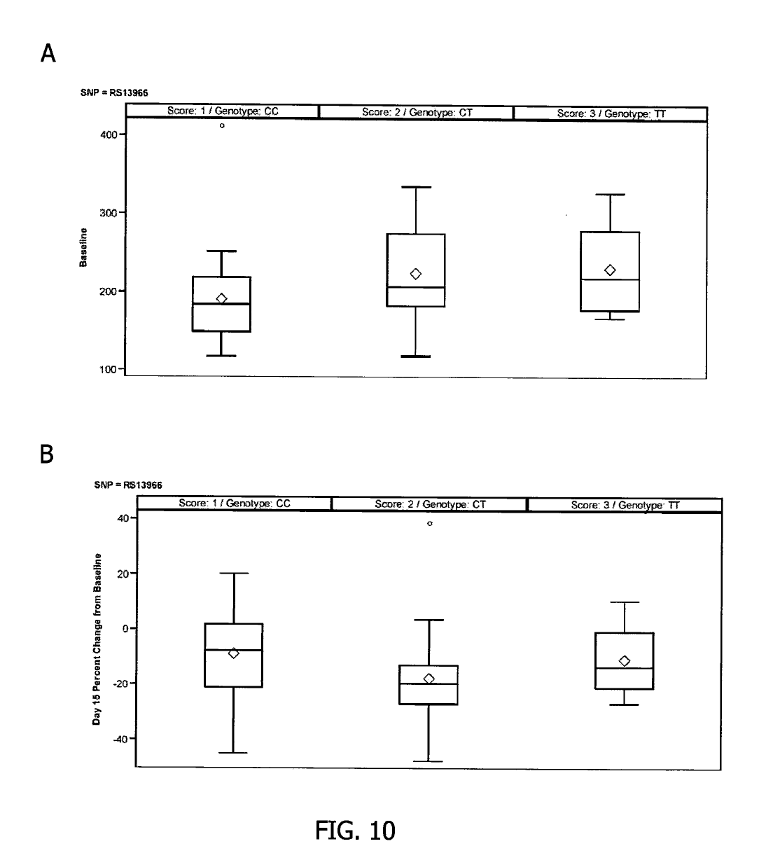

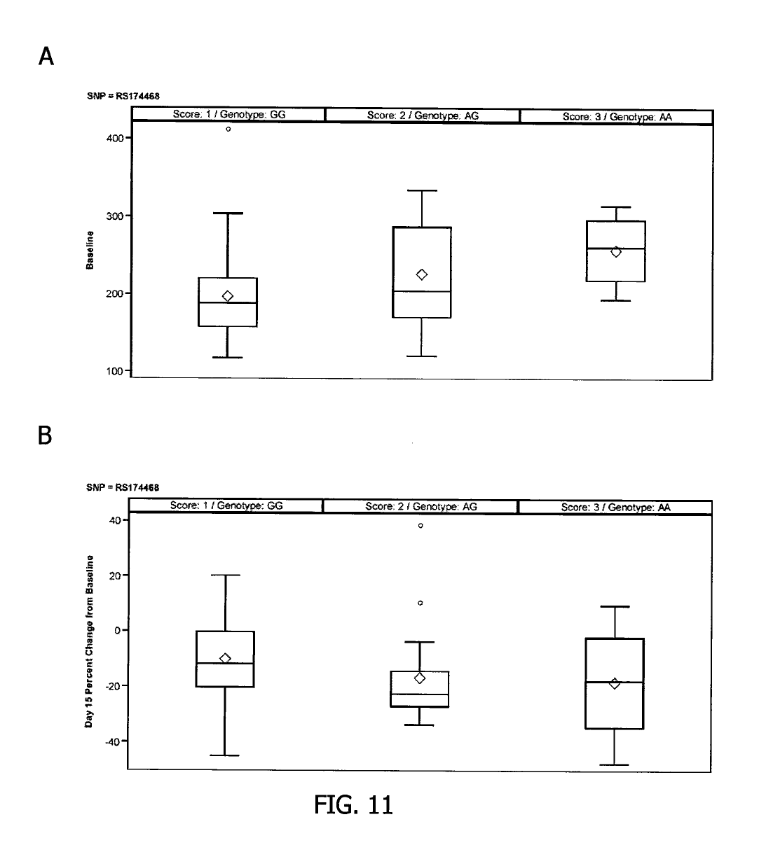

FIG. 10 (rs174589, FADS gene cluster) and FIG. 11 (rs174579, FADS gene cluster) demonstrate that for certain SNPs, subjects who are homozygous for the major allele, rather than minor allele, have higher average and median baseline AA levels. In these individuals, the plasma level of AA in major allele homozygotes is more responsive to 14-day treatment with 4 g/day Epanova® than are the plasma levels for heterozygotes or those homozygous at the minor allele, those genotypes for which baseline plasma levels were lower. For other SNPs, as shown in FIGS. 12-24, no clear genotypic contribution to baseline and post-, treatment AA levels can be seen.

### 6.3. Example 3

### n-3 FFA Compositions have Unprecedented Potency in Reducing Plasma AA Levels and Elevating EPA/AA Ratios

6.3.1. Drug Agents

STUDY DRUG (Epanova®)—Type A porcine soft gelatin capsules were prepared, each containing one gram (1 g) of a PUFA composition comprising omega-3 PUFAs in free acid form (“API”). The capsules were coated with Eudragit NE 30-D (Evonik Industries AG). The API had the composition given in Table 2 (see Example 2, above).

PLACEBO—Capsules were prepared containing olive oil for use as a control.

6.3.2. Study Design

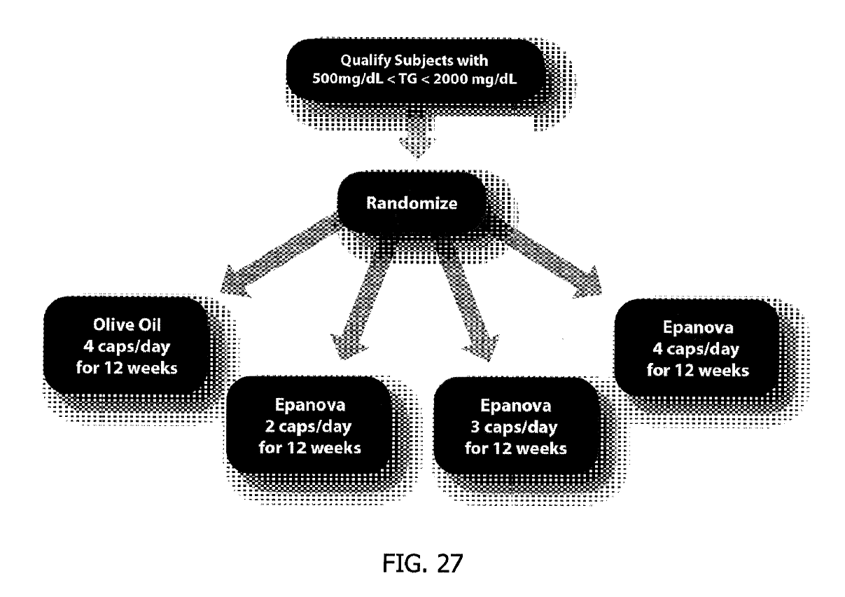

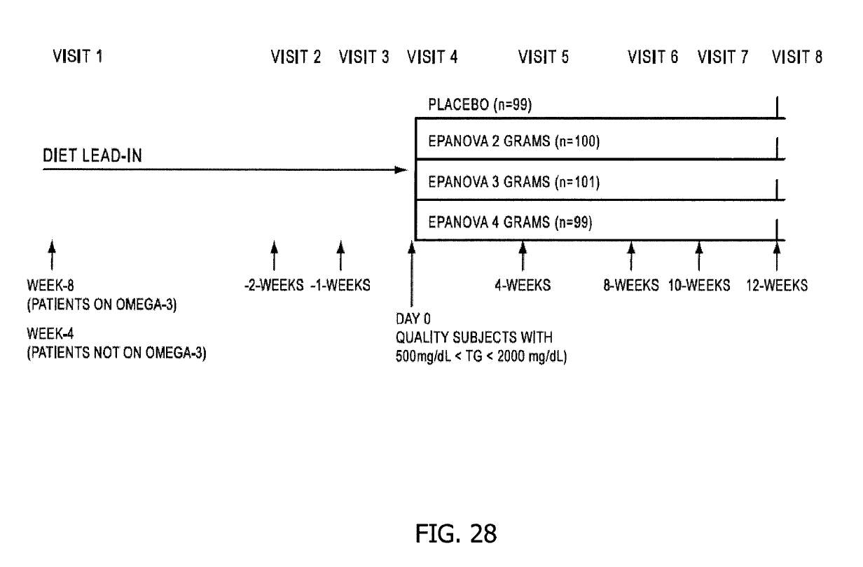

A 12-week double-blind, olive oil-controlled, study was performed in the United States, Denmark, Hungary, India, Netherlands, Russia, and Ukraine. Subjects were selected on the basis of high triglyceride levels, in the range of 500-2,000 mg/dL. Subjects were randomly selected to receive 2, 3, or 4 grams of Epanova®, or 4 grams of olive oil as placebo. The general trial design is illustrated in FIG. 27, with FIG. 28 providing a more detailed treatment flow diagram further identifying the timing of study visits. The primary study endpoint was percent change in plasma triglyceride levels from baseline to end-of-treatment (“EOT”). The secondary endpoint was percent change in plasma non-HDL cholesterol (“non-HDL-c”) from baseline to EOT.

6.3.3. Results

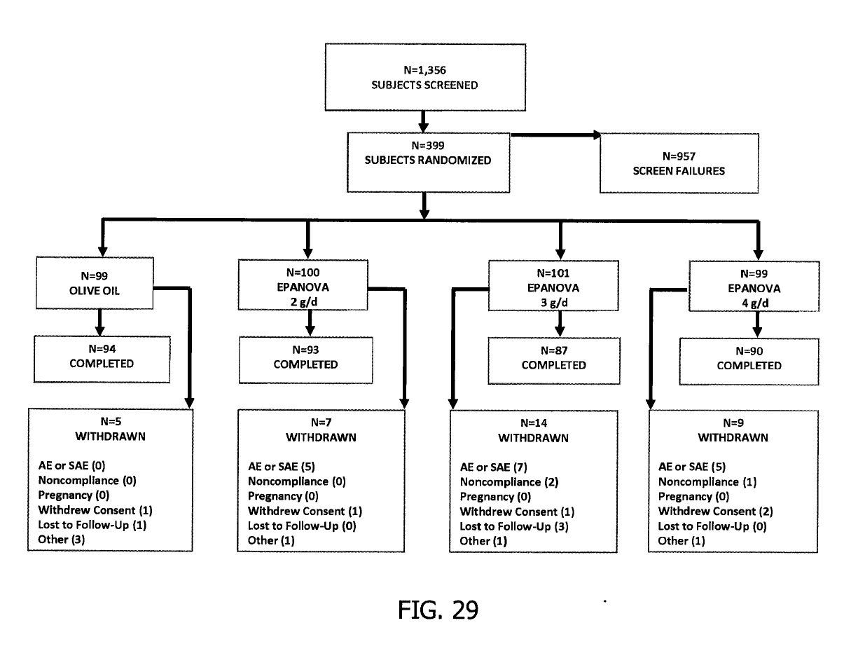

FIG. 29 shows the disposition of all subjects, with “AE” abbreviating “adverse event” and “SAE” abbreviating “serious adverse event.”

A total of 1,356 subjects were initially screened, and of these, 399 were selected to participate in the study. Of the 399 subjects, 99 received olive oil placebo, 100 received Epanova® 2 g/day; 101 received Epanova® 3 g/day; and 99 received Epanova® 4 g/day. Table 4 shows average triglyceride (TG) and cholesterol measurements for the subjects at randomization (prior to treatment), in comparison to desirable levels as described by the Third Report of the Expert Panel on Detection, Evaluation, and Treatment of High Blood Cholesterol in Adults (Adult Treatment Panel III), produced by the National Heart Lung and Blood Institute.

TABLE 4

| | Desirable | Patients randomized for trial | | | |
|---|---|---|---|---|---|
| Parameter | (mg/dL)¹ | 4 g/day | 3 g/day | 2 g/day | Olive Oil |
| TG | <150 | 655 | 715 | 717 | 686 |
| HDL-C | >40 | 29 | 28 | 27 | 29 |
| LDL-C | <100 | 90 | 81 | 77 | 78 |
| Non-HDL-C | <130 | 225 | 215 | 205 | 215 |
| VLDL-C | <30 | 126 | 124 | 123 | 125 |

¹NCEP ATP III, September 2002

Of the patients receiving olive oil, five total were withdrawn from the study due to the following reasons: withdrawn consent (1), lost to follow-up (1), and other reasons (3). Of the patients receiving Epanova 2 g/day, seven total were withdrawn due to the following reasons: adverse effects (5), withdrawn consent (1), and other reasons (1). Of the patients receiving Epanova® 3 g/day, 14 total were withdrawn due to: adverse effects (7), noncompliance (2), withdrawn consent (1), lost to follow-up (3), and other reasons (1). Of the patients receiving Epanova® 4 g/day, 9 were withdrawn, due to: adverse effects (5), noncompliance (1), withdrawn consent (2), and other reasons (1).

Epanova® achieved the primary endpoint of triglyceride reduction and the secondary endpoint of non-HDL cholesterol (total cholesterol level minus the level of HDL-cholesterol) (“non-HDL-C”) reduction at all doses, and produced statistically significant reductions in multiple established markers of atherogenicity: Apo B, Apo CIII, RLP, and LpPLA2 (data not shown). In patients on concomitant statin therapy, Epanova® provided additive efficacy on key lipid parameters: TG; non-HDL-C; HLD-c; total cholesterol (TC); and TC/HDL-C (data not shown).

Plasma levels of EPA, DHA, and DPA—the three species of omega-3 lc-PUFA in greatest abundance in Epanova®—were measured at baseline and at end-of treatment (EOT), as were plasma levels of the omega-6 lc-PUFA, arachidonic acid (AA). Table 5, below, separately tabulates average and median baseline and end-of-treatment (EOT) plasma levels (in μg/mL) for EPA, DHA, DPA, and AA.

TABLE 5
(Baseline and EOT absolute plasma levels)

| | |
|---|---|
| Baseline | EOT |

Average

| | | | |
|---|---|---|---|
| EPA | 2 g | 36.6 | 126.8 |
| | 3 g | 41.4 | 174.7 |
| | 4 g | 38.9 | 199.7 |
| DHA | 2 g | 106.6 | 159.9 |
| | 3 g | 113.7 | 183.6 |
| | 4 g | 104.8 | 188.8 |
| DPA | 2 g | 37.6 | 61.77 |
| | 3 g | 38.71 | 69.36 |
| | 4 g | 36.84 | 69.73 |
| AA | 2 g | 377.9 | 327.4 |
| | 3 g | 394.9 | 344 |
| | 4 g | 393.9 | 298.1 |

Median

| | | | |
|---|---|---|---|
| EPA | 2 g | 26.7 | 104 |
| | 3 g | 30.7 | 141.9 |
| | 4 g | 25.7 | 170 |
| DHA | 2 g | 93.5 | 148.3 |
| | 3 g | 97.4 | 156.9 |
| | 4 g | 91.8 | 169.1 |
| DPA | 2 g | 35.23 | 54.59 |
| | 3 g | 34.71 | 58.56 |
| | 4 g | 32.53 | 66.03 |
| AA | 2 g | 358.4 | 279.2 |
| | 3 g | 368.8 | 313.8 |
| | 4 g | 363.4 | 274.2 |

Baseline plasma levels of EPA, DHA, DPA, and AA indicate effective randomization of subjects among the treatment arms. EPA:AA ratios at baseline were about 0.10 (see Table 8, below).

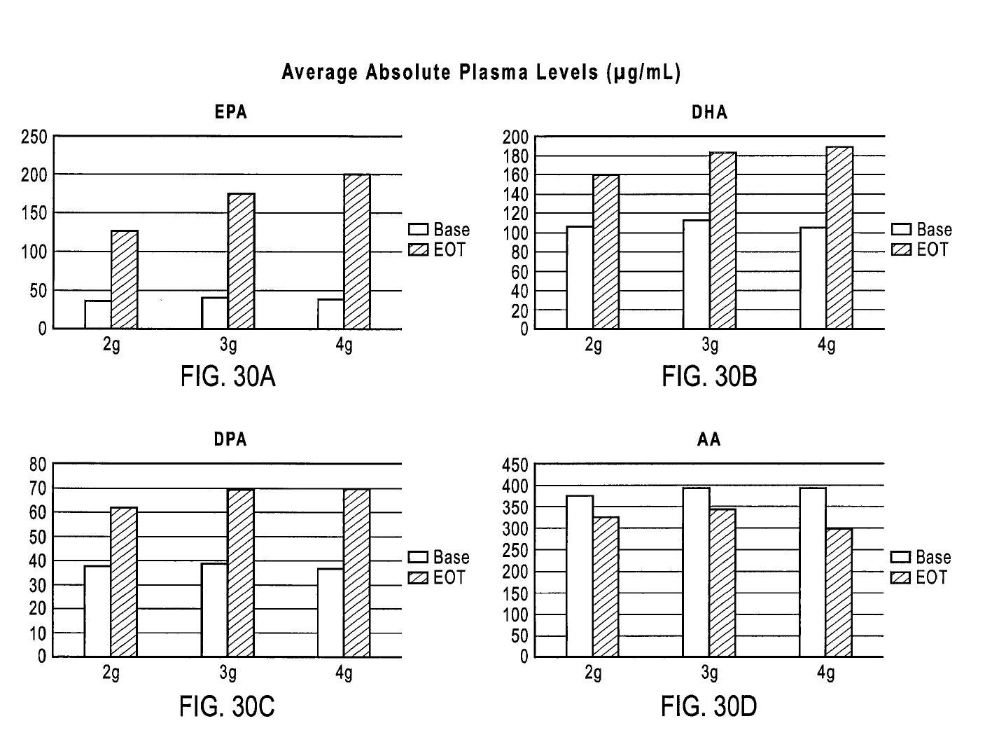

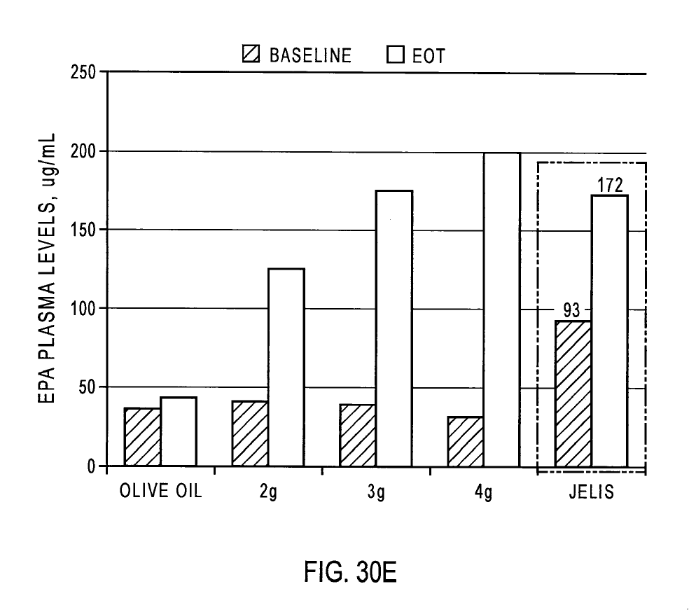

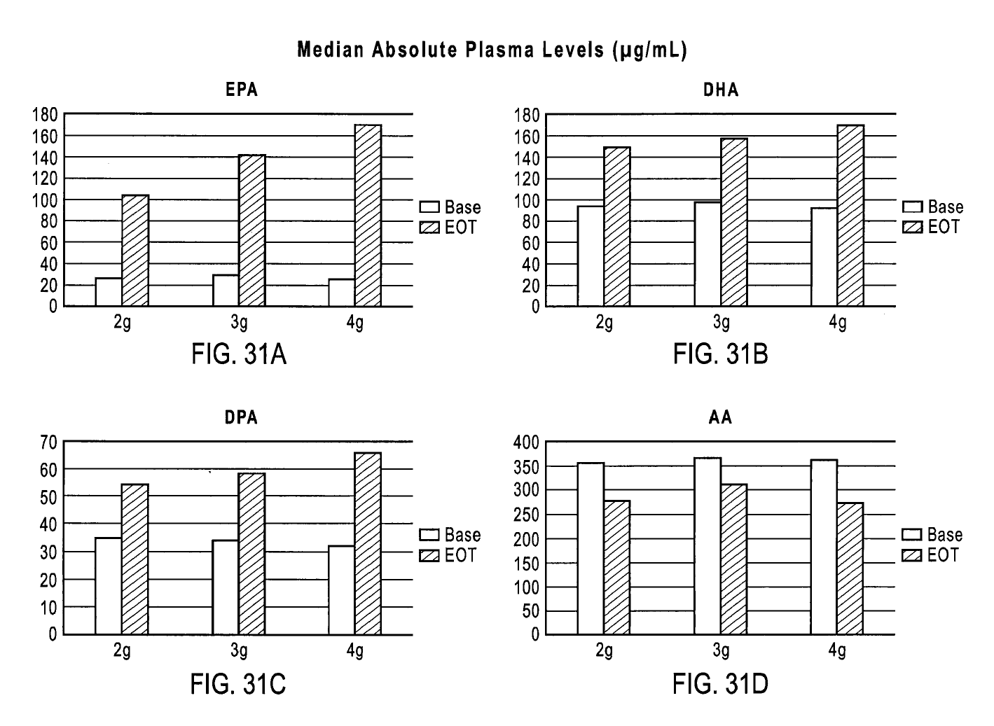

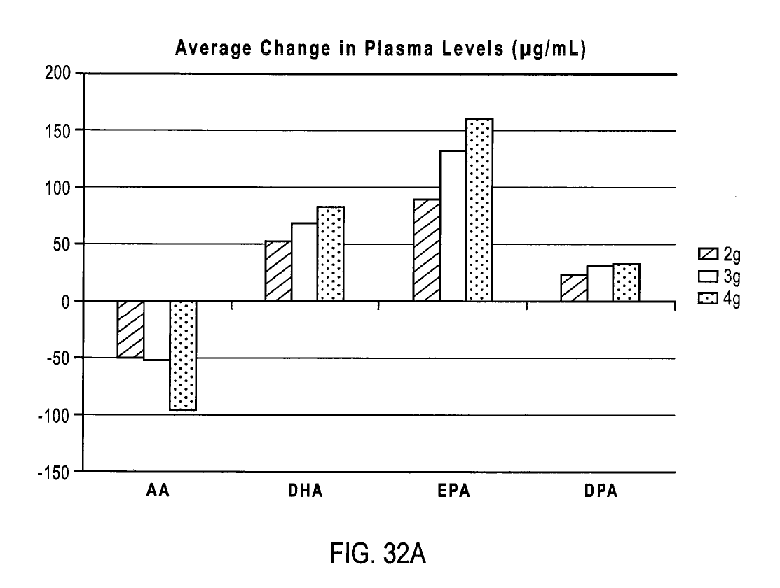

FIGS. 30A-30E plot the average baseline and end-of-treatment (EOT) plasma levels (in μg/mL) for EPA (FIG. 30A), DHA (FIG. 30B), DPA (FIG. 30C) and AA (FIG. 30D), for each of the treatment arms in the EVOLVE trial. FIG. 30E compares average baseline and EOT EPA levels for each treatment arm and the control (olive oil) arm to values earlier reported for the unrelated JELIS trial (“JELIS”). Note that the Japanese subjects in the JELIS trial had higher baseline EPA levels. FIGS. 31A-31D plot median baseline and end-of-treatment (EOT) plasma levels (in μg/mL) for EPA (FIG. 31A), DHA (FIG. 31B), DPA (FIG. 31C), and AA (FIG. 31D).

Table 6, below, tabulates the average change and the median change in absolute plasma levels (in μg/mL) from baseline to EOT for EPA, DHA, DPA, and AA.

TABLE 6
(absolute change in plasma levels)

| | | | |
|---|---|---|---|
| AA | EPA | DPA | DHA |

average change from baseline to EOT (μg/mL)

| | | | | |
|---|---|---|---|---|
| 2 g | −50.5 | 90.2 | 24.17 | 53.3 |
| 3 g | −50.9 | 133.3 | 30.65 | 69.9 |
| 4 g | −95.8 | 160.8 | 32.89 | 84 |

median change from baseline to EOT (μg/mL)

| | | | | |
|---|---|---|---|---|
| 2 g | −79.2 | 77.3 | 19.36 | 54.8 |
| 3 g | −55 | 111.2 | 23.85 | 59.5 |
| 4 g | −89.2 | 144.3 | 33.5 | 77.3 |

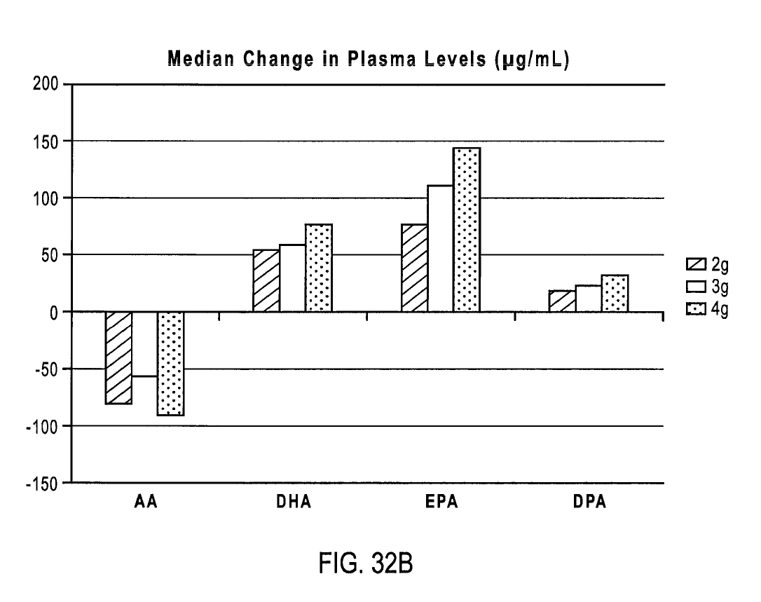

FIGS. 32A and 32B plot the data in the table above, showing the change from baseline to EOT in absolute plasma levels (in μg/mL) of AA, DHA, EPA, and DPA for each of the treatment arms of the EVOLVE trial, with FIG. 32A plotting average change and FIG. 32B showing median change from baseline.

Table 7, below, separately tabulates percentage change from baseline to EOT in the average and median plasma levels of EPA, DHA, DPA, and AA. Table 7 also presents LS mean change (%) for EPA, DHA, and AA.

TABLE 7

| | | | |
|---|---|---|---|
| AA | EPA | DPA | DHA |

average percentage change from baseline to EOT

| | | | | |
|---|---|---|---|---|
| 2 g | −10.5 | 410.8 | 86.08 | 69 |
| 3 g | −11.2 | 538.1 | 96.59 | 88.4 |
| 4 g | −18 | 778.3 | 131.66 | 106 |

median percentage change from baseline to EOT

| | | | | |
|---|---|---|---|---|
| 2 g | −15.6 | 253.9 | 75.3 | 61.2 |
| 3 g | −17.9 | 317 | 68.6 | 61.9 |
| 4 g | −25.9 | 404.8 | 74.87 | 65.5 |

LS mean change (%)

| | | | | |
|---|---|---|---|---|
| 2 g | −15.14 | 267.04 | — | 56.72 |
| 3 g | −15.98 | 331.86 | — | 64.07 |
| 4 g | −23.2 | 406.32 | — | 71.77 |

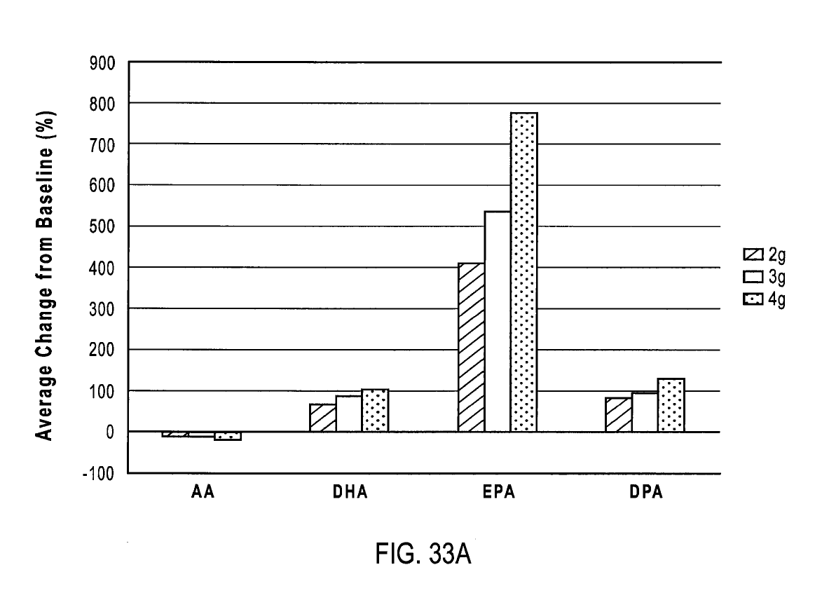

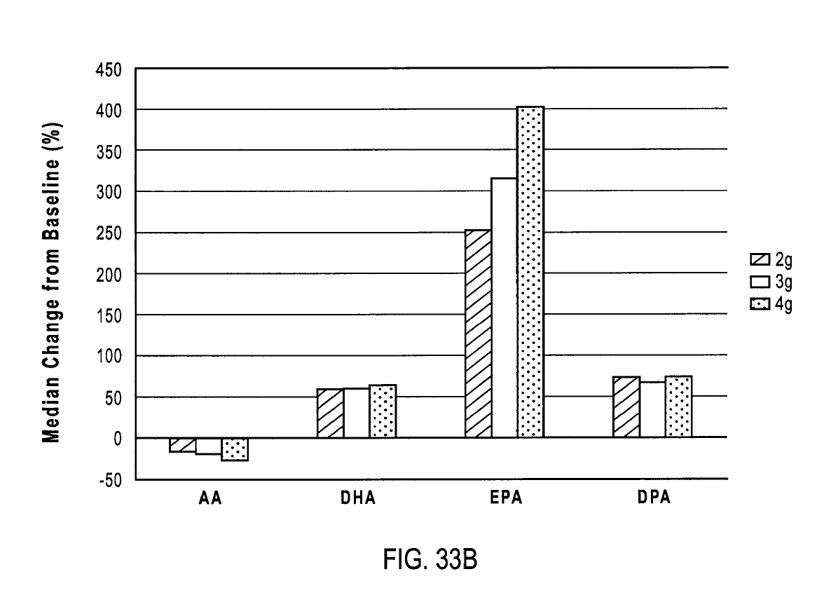

FIG. 33A plots the average change from baseline to EOT, as percentage of baseline value, for AA, DHA, EPA, and DPA in each of the treatment arms of the EVOLVE trial, and FIG. 33B plots the median percent change from baseline to EOT.

Table 8 below presents EPA/AA ratios at beginning and end-of-treatment for each of the treatment arms of the EVOLVE trial.

TABLE 8
EPA/AA ratios

| | |
|---|---|
| baseline | EOT |

average

| | | |
|---|---|---|
| 2 g | 0.096851 | 0.387294 |
| 3 g | 0.104837 | 0.507849 |
| 4 g | 0.098756 | 0.669909 |

median

| | | |
|---|---|---|
| 2 g | 0.074498 | 0.372493 |
| 3 g | 0.083243 | 0.452199 |
| 4 g | 0.070721 | 0.619985 |

As can be seen from Tables 5-7 and FIGS. 30-33, 12 week treatment with Epanova® caused dramatic increases in plasma levels of EPA, DHA, and DPA. For example, at the 2 g dose, the average percentage change from baseline to EOT in EPA plasma levels was 411%; at the 4 g dose, 778%. Median percentage change in EPA plasma levels were respectively 254% and 405%. At the 2 g dose, the average percentage change from baseline to EOT in DHA plasma levels was 69%; at the 4 g dose, the average percentage change was 106%. Median percentage change in DHA plasma levels appear less dramatic, with a 61.2% change at 2 g Epanova®, and 65.5% change at 4 g.

Increases in plasma levels of EPA, DHA, and DPA were accompanied by significant reductions in plasma AA levels, with the 4 g dosage regimen effecting an average reduction of 95.8 μg/mL and median reduction of 89.2 μg/mL, which correspond to an average percentage reduction of 18%, a median percentage change of 25.9%, and a LS mean change of 23.2%. It should be noted that the decrease in plasma arachidonic acid levels was observed despite administering arachidonic acid, which was present at 2.446% (a/a) in the Epanova® batch used in this trial.

The increase in EPA plasma levels and concomitant reduction in AA plasma levels cause a significant improvement in the EPA/AA ratio, as shown in Table 8, from approximately 0.10 at baseline to approximately 0.67 (average) and 0.62 (median) at EOT at the 4 g dose.

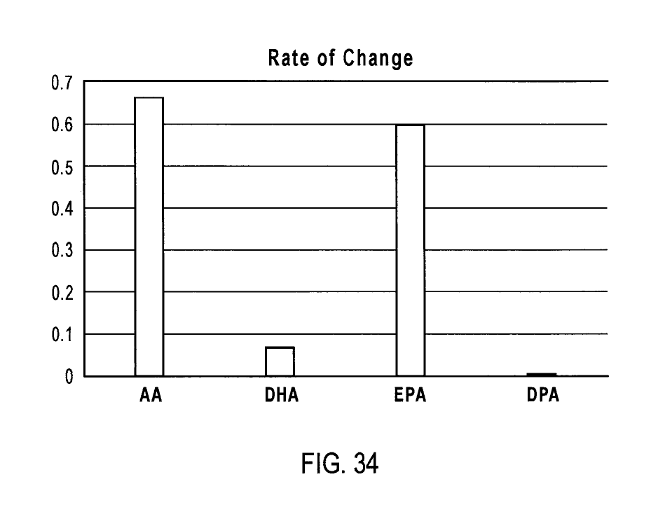

The extremely high bioavailability of the omega-3 PUFAs in Epanova revealed differences in pK response among the omega-3 species, and arachidonic acid. FIG. 34 plots the rate of change in the median percentage change from baseline in plasma levels of EPA, DHA, DPA, and AA (absolute value) between 2 g and 4 g doses of EPANOVA. Table 9, below, tabulates the results:

TABLE 9
(rate of change in median percentage change from baseline)
(absolute value)

| EPA | DHA | DPA | AA |
|---|---|---|---|
| 0.594328476 | 0.070261438 | 0.005710491 | 0.66025641 |

Given little or no increase in plasma levels of DHA and DPA upon doubling of the Epanova® dose from 2 g to 4 g per day, the rate of change (slope) in the median percentage change from baseline is near zero, predicting little further increase in DHA and DPA plasma levels will be seen upon further increase in dose. Similar plateauing of response is seen in triglyceride levels, HDL-c levels, and non-HDL-c levels (data not shown).

By contrast, the rate of change for EPA remains high, with a slope of 0.59; further increase in EPA plasma levels is expected to be obtained by increasing Epanova® dosage above 4 g/day. Significantly, the rate of change in AA levels upon doubling the Epanova® dose from 2 g to 4 g per day is even higher than that for EPA; further reductions in AA plasma levels are expected as Epanova® dosage is increased above 4 g/day. Epanova® thus exhibits unprecedented potency in ability to reduce AA levels.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method for treating, reversing, inhibiting, or preventing resistance to antiplatelet therapy in a subject who is an efficient converter and for whom antiplatelet therapy is clinically indicated, comprising:
administering to the subject an effective amount of a composition comprising omega-3 lc-PUFAs (“omega-3 composition”).

2. The method of claim 1, further comprising the prior step of determining whether the subject is an efficient converter.

3. The method of claim 2, wherein determining whether the subject is an efficient converter comprises determining the subject's genotype at one or more polymorphisms associated with one or more genes selected from the group consisting of FADS1, FADS2, and FADS3.

4. The method of claim 2, wherein determining whether the subject is an efficient converter comprises measuring the level of arachidonic acid in a sample from the subject.

5. The method of claim 1, wherein the amount of omega-3 composition is effective to reduce arachidonic acid (AA) concentration in plasma by at least about 5%.

6. The method of claim 5, wherein the amount of omega-3 composition is effective to reduce plasma AA concentration by at least about 10%.

7. The method of claim 6, wherein the amount of omega-3 composition is effective to reduce plasma AA concentration by at least about 20%.

8. The method of claim 1, wherein the amount of omega-3 composition is effective to reduce plasma arachidonic acid concentration by at least about 50 μg/mL.

9. The method of claim 8, wherein the amount of omega-3 composition is effective to reduce plasma arachidonic acid concentration by at least about 75 μg/mL.

10. The method of claim 1, wherein the amount of omega-3 composition is effective to increase plasma EPA/AA ratio to at least about 0.25.

11. The method of claim 10, wherein the amount of omega-3 composition is effective to increase plasma EPA/AA ratio to at least about 0.50.

12. The method of claim 11, wherein the amount of omega-3 composition is effective to increase plasma EPA/AA ratio to at least about 0.65.

13. The method of claim 1, wherein the omega-3 composition is an n-3 FFA composition.

14. The method of claim 12, wherein the omega-3 composition is an n-3 FFA composition.

15. The method of claim 13, wherein the n-3 FFA composition comprises at least 50% (a/a) EPA.

16. The method of claim 15, wherein the n-3 FFA composition further comprises at least 15% (a/a) DHA.

17. The method of claim 16, wherein the n-3 FFA composition further comprises at least 2.5% (a/a) DPA.

18. The method of claim 1, wherein the amount of omega-3 composition is no more than 4 g/day.

19. The method of claim 18, wherein the amount of omega-3 composition is no more than 2 g/day.

20. The method of claim 19, wherein the omega-3 composition is an n-3 FFA composition.

21. A method of providing antiplatelet therapy to subjects in need thereof, comprising:
(a) determining whether the subject is an efficient converter; and
(b) in those subjects determined to be efficient converters, adjunctively administering (i) an effective amount of an omega-3 composition effective, and (ii) an effective amount of an antiplatelet agent.

22. In a method of providing antiplatelet therapy to subjects in need thereof, the improvement comprising:
(a) determining whether the subject is an efficient converter; and
(b) in those subjects determined to be efficient converters of mc-PUFA to lc-PUFA, adjunctively administering an effective amount of an omega-3 composition.

23. The method of claim 21, wherein determining whether the subject is an efficient converter comprises determining the subject's genotype at one or more polymorphisms associated with one or more genes selected from the group consisting of FADS1, FADS2, and FADS3.

24. The method of claim 21, wherein determining whether the subject is an efficient converter comprises measuring the level of arachidonic acid in a sample from the subject.

25. The method of claim 21, wherein the amount of omega-3 composition is effective to reduce arachidonic acid (AA) concentration in plasma by at least about 5%.

26. The method of claim 25, wherein the amount of omega-3 composition is effective to reduce plasma AA concentration by at least about 10%.

27. The method of claim 26, wherein the amount of omega-3 composition is effective to reduce plasma AA concentration by at least about 20%.

28. The method of claim 21, wherein the amount of omega-3 composition is effective to reduce plasma arachidonic acid concentration by at least about 50 μg/mL.

29. The method of claim 28, wherein the amount of omega-3 composition is effective to reduce plasma arachidonic acid concentration by at least about 75 μg/mL.

30. The method of claim 21, wherein the amount of omega-3 composition is effective to increase plasma EPA/AA ratio to at least about 0.25.

31. The method of claim 10, wherein the amount of omega-3 composition is effective to increase plasma EPA/AA ratio to at least about 0.50.

32. The method of claim 31, wherein the amount of omega-3 composition is effective to increase plasma EPA/AA ratio to at least about 0.65.

33. The method of claim 21, wherein the omega-3 composition is an n-3 FFA composition.

34. The method of claim 22, wherein the omega-3 composition is an n-3 FFA composition.

35. The method of claim 33, wherein the n-3 FFA composition comprises at least 50% (a/a) EPA.

36. The method of claim 35, wherein the n-3 FFA composition further comprises at least 15% (a/a) DHA.

37. The method of claim 36, wherein the n-3 FFA composition further comprises at least 2.5% (a/a) DPA.

38. The method of claim 21, wherein the amount of omega-3 composition is no more than 4 g/day.

39. The method of claim 38, wherein the amount of omega-3 composition is no more than 2 g/day.

40. The method of claim 21, wherein the antiplatelet agent is selected from the group consisting of clopidogrel bisulfate and aspirin.

41. The method of claim 40, wherein the antiplatelet agent is clopidogrel bisulfate.

42. A method of treating a patient with an antiplatelet agent, comprising:
(a) administering a therapeutically effective amount of an inhibitor of platelet aggregation; and
(b) adjunctively administering an effective amount of n-3 FFA composition.

43. In a method of treating a patient with an antiplatelet agent, the improvement comprising:
adjunctively administering an effective amount of an n-3 FFA composition.

44. The method of claim 42, wherein the amount of n-3 FFA composition is effective to reduce arachidonic acid (AA) concentration in plasma by at least about 5%.

45. The method of claim 44, wherein the amount of n-3 FFA composition is effective to reduce plasma AA concentration by at least about 10%.

46. The method of claim 45, wherein the amount of n-3 FFA composition is effective to reduce plasma AA concentration by at least about 20%.

47. The method of claim 42, wherein the amount of n-3 FFA composition is effective to reduce plasma arachidonic acid concentration by at least about 25 μg/mL.

48. The method of claim 47, wherein the amount of n-3 FFA composition is effective to reduce plasma arachidonic acid concentration by at least about 50 μg/mL.

49. The method of claim 48, wherein the amount of n-3 FFA composition is effective to reduce plasma arachidonic acid concentration by at least about 75 μg/mL.

50. The method of claim 42, wherein the amount of n-3 FFA composition is effective to increase plasma EPA/AA ratio to at least about 0.25.

51. The method of claim 50, wherein the amount n-3 FFA composition is effective to increase plasma EPA/AA ratio to at least about 0.50.

52. The method of claim 51, wherein the amount of n-3 FFA composition is effective to increase plasma EPA/AA ratio to at least about 0.65.

53. The method of claim 42, wherein the n-3 FFA composition comprises at least 50% (a/a) EPA.

54. The method of claim 53, wherein the n-3 FFA composition further comprises at least 15% (a/a) DHA.

55. The method of claim 36, wherein the n-3 FFA composition further comprises at least 2.5% (a/a) DPA.

56. The method of claim 42 or claim 43, wherein the n-3 FFA composition comprises about 55% EPA (a/a), about 20% DHA (a/a), and about 5% DPA (a/a).

57. The method of claim 42, wherein the amount of n-3 FFA composition is no more than 4 g/day.

58. The method of claim 57, wherein the amount of n-3 FFA composition is no more than 2 g/day.

59. The method of claim 42, wherein the antiplatelet agent is selected from the group consisting of clopidogrel bisulfate and aspirin.

60. The method of claim 59, wherein the antiplatelet agent is clopidogrel bisulfate.

61. A unit dosage form, comprising:
an omega-3 composition; and
an anti-platelet agent,
wherein the omega-3 composition is contained within a capsule, and the anti-platelet agent is coated on the exterior said capsule.

62. The unit dosage form of claim 61, wherein the anti-platelet agent is clopidogrel bisulfate or aspirin.

63. The unit dosage form of claim 62, wherein the anti-platelet agent is clopidogrel bisulfate.

64. The unit dosage form of claim 61, in which at least 0.5 g of omega-3 composition is encapsulated.

65. The unit dosage form of claim 64, in which at least 1 g of omega-3 composition is encapsulated.

66. The unit dosage form of claim 61, wherein the omega-3 composition is an n-3 FFA composition.

67. The unit dosage form of claim 66, wherein the n-3 FFA composition comprises at least 50% (a/a) EPA.

68. The unit dosage form of claim 67, wherein the n-3 FFA composition further comprises at least 15% (a/a) DHA.

69. The unit dosage form of claim 68, wherein the n-3 FFA composition further comprises at least 2.5% (a/a) DPA.

70. The unit dosage form of claim 66, wherein the capsule is a porcine type A soft gelatin capsule.

71. The unit dosage form of claim 70, wherein the capsule further comprises a coating interposed between the gelatin and the coating comprising the anti-platelet agent.

72. The unit dosage form of claim 71, wherein the interposed coating is capable of delaying release of the n-3 FFA composition for at least 30 minutes at 37° C. in aqueous medium in vitro.

73. The unit dosage form of claim 71, wherein the interposed coating is a neutral poly(ethylacrylate-methylmethacrylate) polymer.
